(19) United States
(12) Patent Application Publication
(10) Pub. No.: US 2026/0185045 A1
(43) Pub. Date: Jul. 2, 2026

(54) COMPOSITIONS AND METHODS FOR GENERATING POLARITY BIASED ORGANOID TISSUE

(71) Applicant: The Regents of the University of Michigan, Ann Arbor, MI (US)

(72) Inventors: Andrew M. Tidball, Ann Arbor, MI (US); Jason R. Spence, Ann Arbor, MI (US)

(21) Appl. No.: 19/434,811

(22) Filed: Dec. 29, 2025

Related U.S. Application Data
(60) Provisional application No. 63/740,109, filed on Dec. 30, 2024.

(51) Int. Cl. C12N 5/079; C12N 5/071; C12N 5/074

(52) U.S. Cl. CPC C12N 5/0618; C12N 5/0679; C12N 5/0688; C12N 5/0696; C12N 2500/36

(57) ABSTRACT

The present disclosure relates to compositions and methods for generating organoids having apical-out polarity or apical-in polarity. In particular, the disclosure provides methods for controlling the polarity (i.e. apical-out or apical-in) of organoids through culturing a population of cells (e.g., patient derived organoids) (e.g., undifferentiated organoid tissue) (e.g., pluripotent stem cell (PSC) derived organoids). To direct apical-out organoids, the disclosure provides methods where an organoid medium is used comprising one or both of lysophosphatidic acid (LPA) and sphingosine-1-phosphate (S1P). To direct apical-in organoids, the disclosure provides methods through culturing a population of cells (e.g., patient derived organoids) (e.g., undifferentiated organoid tissue) (e.g., PSC derived organoids) with an organoid medium lacking LPA and S1P.

### CROSS REFERENCE TO RELATED APPLICATION

The present application claims priority to U. S. Provisional Application No. 63/740,109, filed Dec. 30, 2024, which is incorporated herein by reference in its entirety.

### STATEMENT REGARDING FEDERALLY SPONSORED RESEARCH OR DEVELOPMENT

This invention was made with government support under HD104901, and HD111089 awarded by the National Institutes of Health. The government has certain rights in the invention.

### FIELD OF THE INVENTION

The present disclosure relates to compositions and methods for generating organoids having apical-out polarity or apical-in polarity. In particular, the disclosure provides methods for controlling the polarity (i.e. apical-out or apical-in) of organoids through culturing a population of cells (e.g., patient derived organoids) (e.g., undifferentiated organoid tissue) (e.g., pluripotent stem cell (PSC) derived organoids). To direct apical-out organoids, the disclosure provides methods where an organoid medium is used comprising one or both of lysophosphatidic acid (LPA) and sphingosine-1-phosphate (S1P). To direct apical-in organoids, the disclosure provides methods through culturing a population of cells (e.g., patient derived organoids) (e.g., undifferentiated organoid tissue) (e.g., PSC derived organoids) with an organoid medium lacking LPA and S1P.

### INTRODUCTION

Apicobasal cell polarity in epithelial tissues is an important step in development. Embedding cultured cells in extracellular matrix (ECM) protein hydrogels, such as Matrigel, provides a 3-dimensional setting that allows the formation of organoids that recapitulate some of the structural and functional hallmarks of many organs and tissues. Integrin signaling from the ECM provides a basal polarity cue that allows these structures to form primarily into tube or sphere-like cysts that are “apical-in”, often with single or multiple central apical lumens or cysts.

Recently, groups working in intestinal, lung, and retinal organoid systems have developed techniques for generating “apical-out” organoids via the removal of ECM proteins and growth in suspension culture¹⁻³. This paradigm, first shown in 3D MDCK cell culture, leads to an inversion of the apicobasal polarity⁴. These “apical-out” organoids are important for understanding functions that happen at apical surfaces such as nutrient absorption, microbiome-host interaction, air-tissue interface, cilia motility, and apically located ligand-receptor cell signaling. Similar results have been achieved in intestinal organoids using an antibody that blocks the function of beta1 integrin¹, and knockout of the beta1-integrin gene (ITGB1) from primary mammary epithelial cell cysts also results in “apical-out” orientation⁵.

While these results highlighted the importance of integrin signaling as an extrinsic basal signaling cue, none of these studies explain why removing a basal cue would cause re-orientation of the polarity, rather than maintaining the current polarity.

What is needed in the art is the identification of an apical polarity cue shared by multiple tissues.

The present invention addresses this need.

### SUMMARY OF THE INVENTION

Experiments conducted during the course of developing embodiments for the present invention hypothesized that while ECM serves as a basal polarity cue, organoids may also be responding to an apical cue present in the culture media.

To test this hypothesis, experiments were conducted that sought to make apical-out induced pluripotent stem cell (iPSC)-derived brain organoids by generating ECM-free spheres aggregated from dissociated neuroepithelial cells. Under normal culture media conditions used for brain organoids, basal-out structures formed that are typical of brain organoids generated from embryoid bodies. However, with the addition of 1% fetal bovine serum, all organoids became apical-out, suggesting that an extrinsic apical cue present in serum induces apical-out organoid formation. As described in Examples 1-7, the results present clear evidence that phospholipids found in serum, lysophosphatidic acid (LPA) and sphingosine-1-phosphate (S1P), cause apical out formation through activation of a G-protein coupled receptor (GPCR)→RhoA→Rho-kinase (ROCK)→filamentous actin (F-actin) pathway. The results further demonstrate that LPA-dependent apical-out formation occurs at physiological LPA concentrations found in cerebral spinal fluid (CSF). Finally, the results identified LPA-dependent apical-out orientation in patient-derived lung and intestinal organoids, as well as undifferentiated hiPSC spheres, indicating that LPA serves as a critical and widespread apical cue.

Accordingly, the present disclosure relates to compositions and methods for generating organoids having apical-out polarity or apical-in polarity. In particular, the disclosure provides methods for controlling the polarity (i.e. apical-out or apical-in) of organoids through culturing a population of cells (e.g., patient derived organoids) (e.g., undifferentiated organoid tissue) (e.g., pluripotent stem cell (PSC) derived organoids). To direct apical-out organoids, the disclosure provides methods where an organoid medium is used comprising one or both of lysophosphatidic acid (LPA) and sphingosine-1-phosphate (S1P). To direct apical-in organoids, the disclosure provides methods through culturing a population of cells (e.g., patient derived organoids) (e.g., undifferentiated organoid tissue) (e.g., PSC derived organoids) with an organoid medium lacking LPA and S1P.

In certain embodiments, the present invention provides a method of forming apical-out organoids in a culture, comprising: contacting a population of cells with an organoid medium comprising one or both of lysophosphatidic acid (LPA) and sphingosine-1-phosphate (S1P); and culturing the population of cells in the organoid medium to obtain apical-out organoids, wherein an apical surface of at least a portion of the apical-out organoids faces away from a core thereof.

In some embodiments, the population of cells is capable of organizing into a polarized structure. In some embodiments, the population of cells lack apical-basal polarity. In some embodiments, the population of cells lack apical-out polarity. In some embodiments, the population of cells comprise epithelial cells and/or mesenchymal cells lacking apical-basal polarity. In some embodiments, the population of cells lacks cells having epithelial cells having apical-out polarity. In some embodiments, the population of cells comprise undifferentiated organoid tissue. In some embodiments, the population of cells comprise undifferentiated organoid tissue selected from undifferentiated brain organoid tissue, undifferentiated lung organoid tissue, undifferentiated intestinal organoid tissue, and undifferentiated colon epithelial organoid tissue. In some embodiments, the population of cells comprises undifferentiated induced pluripotent stem cells (iPSCs). In some embodiments, the population of cells comprises human induced pluripotent stem cells (hiPSCs). In some embodiments the population of cells comprises iPSC organoids. In some embodiments, the population of cells comprises hiPSC organoids.

In some embodiments, the population of cells comprises undifferentiated pluripotent stem cells and/or epithelial cell types derived from pluripotent stem cells (e.g. neuroepithelial) or patient tissues (e.g. patient-derived intestinal or airway organoids). In some embodiments, the organoid medium lacks extracellular matrix or extracellular matrix protein. In some embodiments, the organoid medium is serum free.

In some embodiments, the population of cells were not cultured in extracellular matrix (ECM).

In some embodiments, culturing the population of cells is under non-adherent conditions. In some embodiments, the culturing with the organoid medium comprising one or both of LPA and S1P results in activation of one or more of a G-protein coupled receptor (GPCR) pathway, a RhoA pathway, a Rho-kinase (ROCK) pathway, and a filamentous actin (F-actin) pathway. In some embodiments, the culturing with the organoid medium comprising one or both of LPA and S1P results in activation of a GPCR→RhoA→ROCK→F-actin pathway.

In some embodiments, the amount of LPA and/or S1P within the organoid medium is between approximately 30 and 300 nM.

In some embodiments, the LPA and/or S1P within the organoid medium is dissolved in dimethylsulfoxide (DMSO) or bovine serum albumin (BSA).

In some embodiments, the obtained apical-out organoids comprise expression of one or more of: ZO1, F-actin, vimentin, TPX2, PAR3, PALS1, cingulin, myosin-IIb, acetylated tublin, N-cadherin, b-cadherin, and Arl13b.

In some embodiments, the obtained apical-out organoids are apical-out brain organoids comprising expression of one or more of: nestin, PAX6, and N-cadherin.

In some embodiments, the obtained apical-out organoids are maintained and expanded.

In some embodiments, the method further comprises culturing the obtained apical-out organoids in the absence of LPA and SIP, wherein the culturing in the absence of LPA and S1P results in obtained apical-in organoids.

In certain embodiments, the present invention provides an organoid medium for generating apical-out organoids, the medium comprising a basal medium and one or both of LPA and S1P.

In some embodiments, the organoid medium is serum-free. In some embodiments, the organoid medium does not contain or come into contact with an added extracellular matrix or extracellular matrix protein.

In certain embodiments, the present invention provides a composition comprising apical-out organoids generated through the any of the methods described herein.

In certain embodiments, the present invention provides a composition comprising apical-in organoids generated through any of the methods descried herein.

In certain embodiments, the present invention provides a kit comprising a population of cells and a composition comprising one or both of LPA and S1P. In some embodiments, the population of cells is hiPSCs and/or hiPSC organoids and/or undifferentiated organoid tissue.

### BRIEF DESCRIPTION OF DRAWINGS

FIG. 1A-M: Lysophosphatidic acid induces apical-out orientation in brain organoids. A Timeline of the adapted SOSR-CO protocol, highlighting the generation of single cell suspensions at day 4 and aggregation using Aggrewell. B-E Confocal images of cryosectioned organoids after 48 hours of exposure to test compounds and immunolabeling for the apical markers ZO1 and f-actin (DNA counterstained with Bisbenzamide). F-M Whole-mount confocal maximum projections (bottom 100 μm) of organoids imaged for ZO1-EGFP to label apical tight junctions. Organoids are day 7 of differentiation. Scale bars are 100 μm.

FIG. 2A-F: Cryosectioned apical-out organoids treated with 1% fetal bovine serum and immunostained for markers of the anterior neural tube. A ZO-1 staining shows apical-out polarization. B Nestin is a neural progenitor marker in the developing brain. C PAX6 is a transcription factor expressed in the dorsal forebrain while N-Cadherin is a CNS specific cadherin found in adherens junctions. D FOXG1 is a telencephalic transcription factor. Cleaved caspase-3 shows low apoptosis in the cells incorporated in the neuroepithelial layer. Unincorporated cells found in the center have a high level of apoptosis. E,F Expression of phospho-vimentin (S55) and TPX2 mainly on the outside indicates mitosis occurring on the apical-out surface, likely due to interkinetic nuclear migration.

FIG. 3: FGF2 has no impact on orientation and little impact on organoid size. Organoids were treated with various concentrations of FGF2 (0-20 ng/mL) and LPA (0-1000 nM). FGF2 had no discernable effect on orientation or size until 20 ng/mL which caused disorganization at all LPA concentrations.

FIG. 4A-W: Structural and Apical Marker Characterization in Organoids. Neuroepithelial cell spheres were treated with LPA (1000 nM B-E; 100 nM G-J,L-W) or vehicle (A,F,K). Wholemount confocal imaging was performed with 20× objective with a total z distance of 100 μm (A,B,F,G,K,L) or 60× objective over 10 μm (C-E, H-J, M-O). Images are maximum z-projections (A-C,F-H, K-M, P-S) or orthogonal sections (D,E,I,J,N,O,T-W) with the apical side up and basal side down. Panels A-E are from a control iPSC WT line generated from foreskin fibroblasts17. Panels F-O are from the commercially available WT iPSC control line RPChiPS8023G1. Panels P-W are from the commercially available iPSC line AICS-0023. Arrowheads highlight tight junctions. All scale bars are 100 μm unless labelled otherwise.

FIG. 5: BSA vs. DMSO vehicle does not change apical-out induction by LPA. Organoids were treated with 100 nM LPA (bottom panels) with either DMSO vehicle or fatty-acid free bovine serum albumin (FAFBSA) and compared to matched non-LPA treated controls (top panels). Scale bars are 100 μm.

FIG. 6A-S: Specificity of lipid-induced apical-out orientation. All images are maximum projections of ZO1-EGFP confocal imaging stacks for the bottom 100 μm of each sphere. A-J Spheres were treated for 48 hours with bovine serum albumin (BSA) vehicle or 1 μM of each indicated phospholipid, including lysophosphatidic acid (LPA), lysophosphatidylcholine (LPC), lysophosphatidylethanolamine (LPE), phosphatidic acid (PA), phosphatidylethanolamine (PE), phosphatidylinositol (PI) phosphatdylserine (PS), sphingosine-1-phosphate (S1P). K,L,O,P Dose-response comparison between LPA and PA and 100, and 1000 nM. M,N,Q,R 100 nM LPA (M,N) or PA (Q,R) were treated with either the PLA2 inhibitor ACA (M,Q) or LPAAT inhibitor lisofylline (LISO; N,R). S A schematic of the PA to LPA conversion, enzymes, and inhibitors. Organoids are day 7 of differentiation. Scale bars are 100 μm.

FIG. 7A-O: LPA-dependency and reversibility of apical-out organization. Panels A-K are maximum projections of ZO1-EGFP confocal imaging stacks for the bottom 100 μm of each sphere. A-I Images of time series with vehicle (BSA) or LPA treatment at 0-96 hours. At 48 hours, some organoids were switched from LPA-into BSA vehicle-containing media (E) or from BSA into LPA (I), with the resulting structure (apical-in or apical-out) determined by the final treatment condition. J Dose response series for LPA for whole organoids with duplicate organoids in the two rows. K Higher magnification views from the panels in J highlight the intermediate effect at 3 nM, the most consistent apical-out structure from 30-300 nM, and obvious loss of ZO1 tight-junctions in some regions with 3000 nM LPA treatment. L Cell segmentation and metrics obtained by CellPose and CellProfiler. M-O Cell segmentation metrics from organoids exposed to 30-3000 nM LPA concentrations. Data shown as means±SD. N=4-5 organoid means for each data point in M-O. Organoids are day 7 of differentiation. Scale bars are 100 μm.

FIG. 8A-G: Bootstrap random forest model of segmented cell metrics for 30-1000 nM LPA treatment. A,B Confusion matrixes for the training data (A) and the validation data of 20% held-out of training (B). C,D Receiver operator curves for each LPA concentration generated by the model. E,F Color legend for (C,D) and area under the curve for each LPA concentration. G The four most instructive features used to build the model. Three of these features are intensity of ZO1-EGFP signal.

FIGS. 9A-Z: LPA and S1P induced apical-out orientation via GPCR/RhoA/ROCK/F-actin. A-U Neuroepithelial spheroids were incubated for 48 hours with vehicle (BSA), 100 nM LPA or S1P and co-incubated with vehicle (A-C), LPAR1 inhibitor (KI16425, 20 μM) (D-F), Rho inhibitor (C3-Transferase, 1 μg/mL) (G-I), ROCK inhibitor (Y27632, 30 μM) (J-L), actin polymerization inhibitor (cytochlasin-B 3 μM) (M-O), serum response factor inhibitor (CCG-1423, 10 μM) (P-R), or Rho agonist (CN03, 1 μg/mL) (S-U). (V) Dose response to LPAR1 selective agonist, UCM-05194. W Phosphorylated cofilin (S3) western blot and GAPDH at 24 hours of exposure to BSA, LPA, or SIP (1000 nM). X Quantification of pCofilin immunoblot normalized to GAPDH. Y Immunoblot quantification at 0.5, 1, 2, and 4 hours of BSA or LPA exposure. Z Schematic for the proposed LPA/S1P signaling pathway necessary for apical-out orientation. Organoids are day 7 of differentiation. Scale bars are 100 μm.

FIG. 10A-B: Rho antagonist and agonist effects on brain organoid polarity and lumen structure. A Organoids were treated with BSA or with 100 or 1000 nM concentrations of LPA or S1P. The bottom row also received 1 μg/mL C3-transferase, a Rho antagonist. C3-transferase blocked the apical out formation for all treatments. B Organoids exposed only to fatty-acid free BSA were also incubated with 1 μg/mL CN03, a cell permeable Rho agonist, in the bottom row. CN03 treatment did not induce apical-out organoids, but it decreased the number of rosettes, increased rosette size and made their shape more spherical.

FIG. 11: High LPA causes increased f-actin in brain organoids from a control iPSC cell line (foreskin-fibroblast origin). The top and bottom are duplicates from two separate brain organoids for each condition.

FIG. 12: Replicates for LPA, S1P, and inhibitors for AICS-0023 line derived brain organoids.

FIG. 13: LPA and inhibitors (1 μg/mL C3-transferase or 30 μM Y27632) were also tested in brain organoids derived from control cell line RPChiPS8023G1 (Reprocell).

FIG. 14A-H: Long-term LPA causes persistent apical-out orientation and improved VZ morphology. A-H′ Organoids were treated with vehicle (A-D′) or 100 nM LPA (E-H′) starting on day 5. Organoids were cryosectioned and immunostained for PAX6 (red), TBR2 (white), ZO1-EGFP (green), and DNA (blue). Magnified views of insets in A-H are shown in A′-H′. Scale bars are 100 μm.

FIG. 15A-H: LPA causes apical-out orientation in non-neural organoid models. A-B′ Small airway organoids removed from ECM were placed in medium with vehicle or 100 nM LPA. Single confocal sections (A-B) and maximum projections (A′-B′) from whole-mount organoids immunostained for apical proteins reveal apical-in orientation in nearly all vehicle treated and apical-out for nearly all LPA treated organoids. C,D Quantification for ZO1/F-actin localization (C) and quantification of motile cilia position by phase microscopy (D). E-F′ Colonoids removed from ECM were placed in medium with vehicle or 100 nM LPA. Single confocal sections (E-F) and maximum projection (E′-F′) from whole-mount organoids immunostained for apical proteins reveal mixed orientation in vehicle treated and apical-out orientation for LPA treated colonoids. G-H′ Undifferentiated hiPSC spheres formed in Aggrewell plates were placed in media with vehicle or 100 nM LPA. Single confocal sections (G-H) and maximum projections (G′-H′) from whole-mount spheres labelled by ZO1-EGFP demonstrated either apical-in for vehicle-treated or apical-out for LPA treated spheres. Statistical analysis in D performed by Chi-squared test with **** meaning p<0.0001. Scale bars are 100 μm.

FIG. 16: LPA causes multilineage human intestinal organoids (HIOs) to be apical out. HIOs were aggregated from single hindgut endoderm cells with LPA either present or absent. These organoids were fixed at either 7 or 14 day and paraffin-embedded followed by sectioning and immunostaining for epithelial markers, E-Cadherin and CDX2.

### DEFINITIONS

As used in the description of the invention and the claims, the singular forms “a,” “an,” and “the” are intended to include the plural forms as well unless the context indicates otherwise.

As used herein, the term “embryonic stem cells (ESCs),” also commonly abbreviated as ES cells, refers to cells that are pluripotent and derived from the inner cell mass of the blastocyst, an early-stage embryo. For purpose of the present invention, the term “ESCs” is used broadly sometimes to encompass the embryonic germ cells as well.

As used herein, the term “pluripotent stem cells (PSCs),” also commonly known as PS cells, encompasses any cells that can differentiate into nearly all cells, i.e., cells derived from any of the three germ layers (germinal epithelium), including endoderm (interior stomach lining, gastrointestinal tract, the lungs), mesoderm (muscle, bone, blood, urogenital), and ectoderm (epidermal tissues and nervous system). PSCs can be the descendants of totipotent cells or obtained through induction of a non-pluripotent cell, such as an adult somatic cell, by forcing the expression of certain genes.

As used herein, the term “induced pluripotent stem cells (iPSCs),” also commonly abbreviated as iPS cells, refers to a type of pluripotent stem cells artificially derived from a normally non-pluripotent cell, such as an adult somatic cell, by inducing a “forced” expression of certain genes. iPSC organoids are organoids derived from iPSCs.

The term “hiPSCs” refers to human induced pluripotent stem cells with similar phenotypical and genotypical characteristics as human embryonic stem cells (hESCs) or human pluripotent stem cells (HPSCs). HiPSCs have self-renewing capabilities similar to hESCs and can undergo three germ layers, producing all the germ layer cells with appropriate growth factors such as endodermal lineage-derived intestinal cells or organoids. hiPSC organoids are organoids derived from hiPSCs.

As used herein, the term “precursor cell” encompasses any cells that can be used in methods described herein, through which one or more precursor cells acquire the ability to renew itself or differentiate into one or more specialized cell types. In some embodiments, a precursor cell is pluripotent or has the capacity to becoming pluripotent. In some embodiments, the precursor cells are subjected to the treatment of external factors (e.g., growth factors) to acquire pluripotency. In some embodiments, a precursor cell can be a totipotent (or omnipotent) stem cell; a pluripotent stem cell (induced or non-induced); a multipotent stem cell; an oligopotent stem cells and a unipotent stem cell. In some embodiments, a precursor cell can be from an embryo, an infant, a child, or an adult. In some embodiments, a precursor cell can be a somatic cell subject to treatment such that pluripotency is conferred via genetic manipulation or protein/peptide treatment.

In developmental biology, cellular differentiation is the process by which a less specialized cell becomes a more specialized cell type. As used herein, the term “directed differentiation” describes a process through which a less specialized cell becomes a particular specialized target cell type. The particularity of the specialized target cell type can be determined by any applicable methods that can be used to define or alter the destiny of the initial cell. Exemplary methods include but are not limited to genetic manipulation, chemical treatment, protein treatment, and nucleic acid treatment.

As used herein, the term “cellular constituents” are individual genes, proteins, mRNA expressing genes, and/or any other variable cellular component or protein activities such as the degree of protein modification (e.g., phosphorylation), for example, that is typically measured in biological experiments (e.g., by microarray or immunohistochemistry) by those skilled in the art. Significant discoveries relating to the complex networks of biochemical processes underlying living systems, common human diseases, and gene discovery and structure determination can now be attributed to the application of cellular constituent abundance data as part of the research process. Cellular constituent abundance data can help to identify biomarkers, discriminate disease subtypes and identify mechanisms of toxicity.

As used herein, the term “organoid” is used to mean a 3-dimensional growth of mammalian cells in culture that retains characteristics of the tissue in vivo, e.g. prolonged tissue expansion with proliferation, multilineage differentiation, recapitulation of cellular and tissue ultrastructure, etc. This disclosure primarily focuses on epithelial organoids, but is not necessarily limited to only epithelial organoids.

As used herein, the term “organoid medium” or “organoid media” refers to a cell culture medium comprising a basal medium that is appropriately supplemented to form the organoids of this disclosure. Indeed, basal media are well known in the art and are routinely formulated to include one or more of salt(s), amino acid(s), carbohydrate(s), buffer(s), trace elements, etc. Examples of commercially available basal media include DMEM, Adv-DMEM, DMEM/F-12, RPMI, Iscoves, and various others marketed specifically for the culture of epithelial cells. The specific supplementation of the basal medium—with for example cytokines, growth factors, small molecules, serum/albumin/serum replacement, lipids, etc.—will depend on the application to which the organoid medium is put. In one embodiment, an organoid medium comprises a basal medium and one or both of lysophosphatidic acid (LPA) and sphingosine-1-phosphate (S1P) for purposes of inducing apical-out organoids from a population of cells (e.g., undifferentiated organoid tissue) (e.g., induced pluripotent stem cells).

As used herein, the term “apical-out” refers to an organoid, and in some embodiments an epithelial organoid, wherein the apical surface is in direct contact with the external environment (i.e. a cell culture medium). Among apical-out organoids, some or all of such organoids may comprise a lumen, in which case a basolateral side thereof is in direct contact with the lumen. However, some apical-out organoids may not comprise a lumen, but may rather exhibit a compact/dense conformation, in which case the basolateral side thereof may be comprised in or adjacent a central core of such apical-out organoids.

As used herein, “differentiate” or “differentiated” are used to refer to the process and conditions by which immature (unspecialized) cells acquire characteristics, becoming mature (specialized) cells, thereby acquiring particular form and function. Stem cells (unspecialized) are often exposed to varying conditions (e.g., growth factors and morphogenic factors) to induce specified lineage commitment, or differentiation, of said stem cells. The process by which an unspecialized (“uncommitted”) or less specialized cell acquires the features of a specialized cell such as, for example, a blood cell or a muscle cell. A differentiated or differentiation-induced cell has taken on a more specialized (“committed”) position within the lineage of a cell. The term “committed”, when applied to the process of differentiation, refers to a cell that has proceeded in the differentiation pathway to a point where, under normal circumstances, it will continue to differentiate into a specific cell type or a subset of cell types, and cannot, under normal circumstances, differentiate into a different cell type or revert to a less differentiated cell type.

As used herein, the terms “genetically modified” or “engineered” cell as used herein refers to a cell into which an exogenous nucleic acid has been introduced by a process involving the hand of man (or a descendant of such a cell that has inherited at least a portion of the nucleic acid). The nucleic acid may for example contain a sequence that is exogenous to the cell, it may contain native sequences (i.e., sequences naturally found in the cells) but in a non-naturally occurring arrangement (e.g., a coding region linked to a promoter from a different gene), or altered versions of native sequences, etc. The process of transferring the nucleic acid into the cell can be achieved by any suitable technique. Suitable techniques include calcium phosphate or lipid-mediated transfection, electroporation, and transduction or infection using a viral vector. In some embodiments the polynucleotide or a portion thereof is integrated into the genome of the cell. The nucleic acid may have subsequently been removed or excised from the genome, provided that such removal or excision results in a detectable alteration in the cell relative to an unmodified but otherwise equivalent cell. It should be appreciated that the term genetically modified is intended to include the introduction of a modified RNA directly into a cell (e.g., a synthetic, modified RNA). Such synthetic modified RNAs include modifications to prevent rapid degradation by endo- and exo-nucleases and to avoid or reduce the cell's innate immune or interferon response to the RNA. Modifications include, but are not limited to, (a) end modifications, e.g., 5′ end modifications (phosphorylation dephosphorylation, conjugation, inverted linkages, etc.) or 3′ end modifications (conjugation, DNA nucleotides, inverted linkages, etc.); (b) base modifications, e.g., replacement with modified bases, stabilizing bases, destabilizing bases, bases that base pair with an expanded repertoire of partners, or conjugated bases; (c) sugar modifications (e.g., at the 2′ position or 4′ position) or replacement of the sugar; and (d) internucleoside linkage modifications, including modification or replacement of the phosphodiester linkages. To the extent that such modifications interfere with translation, the modification is not suitable for the methods and compositions described herein.

### DETAILED DESCRIPTION OF THE INVENTION

Apicobasal polarization is crucial for tissue organization during in vivo development and in human organoid models. Extracellular matrix (ECM) signaling typically provides a basal cue, and human intestinal and lung organoids have been shown to reverse polarity from apical-in to apical-out after ECM removal. However, ECM-free brain organoids maintain apical-in polarity, suggesting that media components may influence polarity. As described in Examples 1-7, exposing brain organoids to serum induced apical-out orientation, even after boiling, and lysophosphatidic acid (LPA) was identified as an apical polarity cue, which is present in the medium of previous apical-out organoid techniques. LPA-directed apical-out formation in brain organoids occurred within a day, lasted at least one month, was reversible, and was optimal at human CSF concentrations of LPA. Sphingosine-1-phosphate (S1P) induced similar apical-out polarization. Pharmacological studies revealed LPA/S1P act via the GPCR/Rho/ROCK/F-actin pathway. Finally, LPA or S1P caused apical-out polarity in patient-derived human lung and intestinal organoids, iPSC spheres, and iPSC-derived human intestinal organoids which contain multiple cell lineages. These findings indicate that LPA and/or S1P signaling is a critical apical polarity cue shared by multiple tissues.

Accordingly, the present disclosure relates to compositions and methods for generating organoids having apical-out polarity or apical-in polarity. In particular, the disclosure provides methods for controlling the polarity (i.e. apical-out or apical-in) of organoids through culturing a population of cells (e.g., patient derived organoids) (e.g., undifferentiated organoid tissue) (e.g., pluripotent stem cell (PSC) derived organoids). To direct apical-out organoids, the disclosure provides methods where an organoid medium is used comprising one or both of lysophosphatidic acid (LPA) and sphingosine-1-phosphate (S1P). To direct apical-in organoids, the disclosure provides methods through culturing a population of cells (e.g., patient derived organoids) (e.g., undifferentiated organoid tissue) (e.g., PSC derived organoids) with an organoid medium lacking LPA and S1P.

In one aspect of this disclosure are provided organoid media for generating apical-out organoids. In some embodiments, the organoid media are for generating undifferentiated apical-out organoids. In some embodiments, the organoid media is for generation tissue specific apical-out organoids (e.g., brain organoids, lung organoids, intestinal organoids, colon organoids, etc.). In some embodiments, the organoid media comprises one or both of LPA and S1P.

If present in the organoid media, the LPA will be present at an effective concentration while also being present at a concentration that does not result in any or in significant levels of cell toxicity. In one embodiment, a concentration of LPA ranges between about 1 nM and 1 mM, between about 5 nM and 500 μM, between about 10 nM and 200 μM, between about 3 nM and 300 nM, between about 50 nM and 100 μM, between about 100 nM and 50 μM, or between about 0.5 μM and 20 μM.

If present in the organoid media, the S1P will be present at an effective concentration while also being present at a concentration that does not result in any or in significant levels of cell toxicity. In one embodiment, a concentration of S1P ranges between about 1 nM and 1 mM, between about 5 nM and 500 μM, between about 10 nM and 200 μM, between about 3 nM and 300 nM, between about 50 nM and 100 μM, between about 100 nM and 50 μM, or between about 0.5 μM and 20 μM.

The organoid media for generating apical-out organoids is capable of activating one or more of a G-protein coupled receptor (GPCR) pathway, a RhoA pathway, a Rho-kinase (ROCK) pathway, and a filamentous actin (F-actin) pathway within a population of cells (e.g., undifferentiated organoid tissue) (e.g., PSCs).

The organoid media for generating apical-out organoids is capable of activating a G-protein coupled receptor (GPCR)→RhoA→Rho-kinase (ROCK)→filamentous actin (F-actin) pathway within a population of cells (e.g., undifferentiated organoid tissue) (e.g., PSCs).

In one embodiment, the organoid medium does not include an extracellular matrix or an extracellular matrix protein. Thus, in one embodiment, the organoid medium does not include (or contain or come into contact with) an added extracellular matrix or extracellular matrix protein. Accordingly, the organoid medium does not require Matrigel™ or any other extracellular matrix to form the apical-out organoids.

In one embodiment, the organoid medium does not contain or come into contact with an extracellular matrix or extracellular matrix protein (unless naturally produced by the cells being cultured). In one embodiment, the organoid does not form in the presence of an exogenously added extracellular matrix or extracellular matrix protein. In one embodiment, the cells (e.g., patient derived organoids) (e.g., undifferentiated organoid tissue) (e.g., PSC derived organoids) do not come into contact with an exogenously added extracellular matrix or extracellular matrix protein(s).

In one embodiment, the organoid medium is serum-free. In some embodiments of a serum-free medium, the medium may comprise an albumin or a different serum replacement. If the organoid medium includes an albumin, it may be isolated from serum, and more specifically from animal serum. Or, the albumin may be recombinant and expressed in a cell line, such as a bacterial, fungal, plant, or animal cell line.

In one embodiment, the organoid medium (e.g., comprising one or both of LPA and S1P) dissolved in dimethylsulfoxide (DMSO) or bovine serum albumin (BSA).

In one aspect of this disclosure are provided methods for generating organoids, a proportion of which are apical-out organoids. In a preferred embodiment, the methods are for generating apical-out tissue specific organoids, such as starting from undifferentiated brain organoid tissue, undifferentiated lung organoid tissue, undifferentiated intestinal organoid tissue, or undifferentiated colon epithelial organoid tissue.

Methods of forming organoids (e.g. apical-out organoids) in a culture will comprise contacting a population of cells (e.g., patient derived organoids) (e.g., undifferentiated organoid tissue) (e.g., PSC derived organoids) with an organoid medium comprising one or both of LPA and S1P. In one embodiment, an organoid medium of this disclosure comprises a basal medium and one or both of LPA and S1P.

Methods of forming organoids (e.g. apical-out organoids) in a culture will comprise culturing the population of cells (e.g., patient derived organoids) (e.g., undifferentiated organoid tissue) (e.g., PSC derived organoids) in the organoid medium comprising one or both of LPA and S1P. In one embodiment, culturing the population of cells (e.g. pulmonary lineage cells) in the organoid medium occurs in the absence of an added extracellular matrix or extracellular matrix protein.

The methods will yield apical-out organoids wherein an apical surface of at least a portion of the apical-out organoids faces away from a core (e.g. a lumen) thereof, but in any event the apical surface of apical-out organoids will be in direct contact with the external environment. In one embodiment, a majority of the formed organoids (i.e the portion) exhibit an apical-out morphology/organization. In one embodiment, among at least 60% of the organoids the apical surface faces away from the core (or, lumen). In one embodiment, among at least 80% of the organoids the apical surface faces away from the core (or, lumen). In one embodiment, about 90% or more of the organoids exhibit an apical surface that faces away from the core (or, lumen) thereof.

In one embodiment, the methods further comprise aggregating the population of cells (e.g., patient derived organoids) (e.g., undifferentiated organoid tissue) (e.g., PSC derived organoids). In one embodiment, the methods further comprise aggregating the population of cells (e.g., patient derived organoids) (e.g., undifferentiated organoid tissue) (e.g., PSC derived organoids) prior to or at the same time as the contacting step. The cells may be aggregated using any known means.

In one embodiment, aggregating the cells (e.g., patient derived organoids) (e.g., undifferentiated organoid tissue) (e.g., PSC derived organoids) occurs in the absence of an added extracellular matrix or extracellular matrix protein(s). In one embodiment, the aggregating and aggregated cells (e.g., patient derived organoids) (e.g., undifferentiated organoid tissue) (e.g., PSC derived organoids) do not contact or come into contact with an added extracellular matrix or extracellular matrix protein(s).

In one embodiment, aggregating the population of cells (e.g., patient derived organoids) (e.g., undifferentiated organoid tissue) (e.g., PSC derived organoids) comprises bringing between 10 and 2000 cells into close proximity in a common well. In one embodiment, the number of cells formed into an aggregate is between 50 and 150 cells. In one embodiment, the deposited cells are single cells or comprised in clumps, or a mixture thereof.

In one embodiment, the population of cells (e.g., patient derived organoids) (e.g., undifferentiated organoid tissue) (e.g., PSC derived organoids) are aggregated in an organoid medium comprising one or both of LPA and S1P. In one embodiment, the population of cells (e.g., patient derived organoids) (e.g., undifferentiated organoid tissue) (e.g., PSC derived organoids) are aggregated in an organoid medium comprising one or both of LPA and S1P for between 1 and 7 days. In one embodiment, the population of cells (e.g., patient derived organoids) (e.g., undifferentiated organoid tissue) (e.g., PSC derived organoids) are aggregated in an organoid medium comprising one or both of LPA and S1P for 4 days+2 days.

In one embodiment, culturing the population of cells (e.g., patient derived organoids) (e.g., undifferentiated organoid tissue) (e.g., PSC derived organoids) is under non-adherent conditions. In one embodiment, the aggregating step is performed under non-adherent conditions, such as through using ultra low attachment 96-well plates. In one embodiment, the contacting step is performed under non-adherent conditions. In one embodiment, the contacting and the culturing steps are under non-adherent conditions, as well as the aggregating step, if taken.

In one embodiment, culturing the population of cells (e.g., patient derived organoids) (e.g., undifferentiated organoid tissue) (e.g., PSC derived organoids) in the organoid medium comprising one or both of LPA and SIP and in the absence of an added extracellular matrix or extracellular matrix protein(s), is for a time sufficient to yield apical-out organoids. In one embodiment, culturing is for between 5 and 25 days. In one embodiment, culturing is for 9 days+3 days. In one embodiment, culturing is for 15 days. In embodiments where the population of cells are aggregated prior to or during the contacting step, the culturing step may be shortened if it is apparent that apical-out organoids are emerging.

As discussed with regard to organoid media of this disclosure (e.g., comprising one or both of LPA and S1P), such organoid media do not include an extracellular matrix or an extracellular matrix protein. In one embodiment, the organoid medium does not contain or come into contact with an extracellular matrix or extracellular matrix protein (unless naturally produced by the cells being cultured). Thus, in one embodiment, the organoid medium does not include (or contain or come into contact with) an added extracellular matrix or extracellular matrix protein. Accordingly, the organoid medium does not require Matrigel™ or any other extracellular matrix to form the apical-out organoids.

Similarly, the contacting, culturing, and if applicable aggregating, steps are performed in the absence of an added extracellular matrix or extracellular matrix protein. Thus, the laboratory ware used during these steps is not coated with an extracellular matrix or an extracellular matrix protein. Accordingly, extracellular matrices commonly used for organoid formation, such as Matrigel™ or others, are not needed to practice the methods of this disclosure.

Apical-out organoids that have been formed in accordance with this disclosure may express one or more of ZO1, F-actin, vimentin, TPX2, PAR3, PALS1, cingulin, myosin-IIb, acetylated tublin, N-cadherin, b-cadherin, and Arl13b.

Apical-out organoids that have been formed in accordance with this disclosure may express one or more of nestin, PAX6, and N-cadherin.

In further aspects, the obtained apical-out organoids may be cultured in the absence of LPA and S1P resulting in obtaining apical-in organoids.

Such methods are not limited to a particular type of population of cells for culturing with the organoid medium comprising one or both of LPA and S1P to obtain apical-out organoids. In some aspects, the population of cells lack apical-basal polarity. In some aspects, the population of cells lack apical-out polarity. In some aspects, the population of cells comprise epithelial cells and/or mesenchymal cells lacking apical-basal polarity. In some/or mesenchymal cells having apical-out polarity.

In some embodiments, the population of cells comprises undifferentiated pluripotent stem cells and/or epithelial cell types derived from pluripotent stem cells (e.g. neuroepithelial) or patient tissues (e.g. patient-derived intestinal or airway organoids).

In some aspects, the population of cells is organoid tissue. As used here, the term “organoid” or “organoid tissue” refers to a three dimensional in vitro model of an organ. An organoid can provide a realistic micro-anatomy of the organ from which it is derived. Accordingly, the term “epithelial organoid” refers to an organoid obtained from one or more epithelial stem/progenitor cells. The epithelial stem/progenitor cell(s) may be from any organ, including but not limited to, the brain, colon, liver, lung, pancreas or intestine. An organoid obtained from one or more brain epithelial stem/progenitor cells is also referred to herein as a “brain organoid”, an organoid obtained from one or more pancreatic epithelial stem/progenitor cells is also referred to herein as a “pancreatic organoid”, an organoid obtained from one or more liver epithelial stem/progenitor cells is also referred to herein as a “liver organoid”, an organoid obtained from one or more colon epithelial stem/progenitor cells is also referred to herein as a “colon organoid” or “colonoid”, and an organoid obtained from one or more intestinal epithelial stem/progenitor cells is also referred to herein as an “intestinal organoid”.

In some aspects, the organoid tissue is undifferentiated organoid tissue. Undifferentiated organoid tissue can be developed into a specific tissue by culturing, for example, with specific growth factors.

In some aspects, the undifferentiated organoid tissue is non-tissue specific. In some aspects, the undifferentiated organoid tissue is selected from undifferentiated brain organoid tissue, undifferentiated lung organoid tissue, undifferentiated intestinal organoid tissue, and undifferentiated colon epithelial organoid tissue.

In some aspects, the population of cells includes stem cells.

In some aspects, the population of cells includes pluripotent stem cells or stem cells that can be induced to become pluripotent. In some aspects, pluripotent stem cells are derived from embryonic stem cells, which are in turn derived from totipotent cells of the early mammalian embryo and are capable of unlimited, undifferentiated proliferation in vitro. Embryonic stem cells are pluripotent stem cells derived from the inner cell mass of the blastocyst, an early-stage embryo. Methods for deriving embryonic stem cells from blastocytes are well known in the art. For example, three cell lines (H1, H13, and H14) have a normal XY karyotype, and two cell lines (H7 and H9) have a normal XX karyotype.

Additional stem cells that can be used include but are not limited to those provided by or described in the database hosted by the National Stem Cell Bank (NSCB), Human Embryonic Stem Cell Research Center at the University of California, San Francisco (UCSF); WISC cell Bank at the Wi Cell Research Institute; the University of Wisconsin Stem Cell and Regenerative Medicine Center (UW-SCRMC); Novocell, Inc. (San Diego, Calif.); Cellartis AB (Goteborg, Sweden); ES Cell International Pte Ltd (Singapore); Technion at the Israel Institute of Technology (Haifa, Israel); and the Stem Cell Database hosted by Princeton University and the University of Pennsylvania. Indeed, embryonic stem cells that can be used in embodiments in accordance with the present invention include but are not limited to SA01 (SA001); SA02 (SA002); ES01 (HES-1); ES02 (HES-2); ES03 (HES-3); ES04 (HES-4); ES05 (HES-5); ES06 (HES-6); BG01 (BGN-01); BG02 (BGN-02); BG03 (BGN-03); TE03 (13); TE04 (14); TE06 (16); UC01 (HSF1); UC06 (HSF6); WA01 (H1); WA07 (H7); WA09 (H9); WA13 (H13); WA14 (H14).

In some aspects, the stem cells are further modified to incorporate additional properties. Exemplary modified cell lines include but not limited to H1 OCT4-EGFP; H9 Cre-LoxP; H9 hNanog-pGZ; H9 hOct4-pGZ; H9 in GFPhES; and H9 Syn-GFP.

More details on embryonic stem cells can be found in, for example, Thomson et al., 1998, Science 282 (5391): 1145-1147; Andrews et al., 2005, Biochem Soc Trans 33:1526-1530; Martin 1980, Science 209 (4458): 768-776; Evans and Kaufman, 1981, Nature 292 (5819): 154-156; Klimanskaya et al., 2005, Lancet 365 (9471): 1636-1641).

Alternatively, pluripotent stem cells can be derived from embryonic germ cells (EGCs), which are the cells that give rise to the gametes of organisms that reproduce sexually. EGCs are derived from primordial germ cells found in the gonadal ridge of a late embryo, have many of the properties of embryonic stem cells. The primordial germ cells in an embryo develop into stem cells that in an adult generate the reproductive gametes (sperm or eggs). In mice and humans it is possible to grow embryonic germ cells in tissue culture under appropriate conditions. Both EGCs and ESCs are pluripotent. For purpose of the present invention, the term “ESCs” is used broadly sometimes to encompass EGCs.

In some aspects, iPSCs are derived by transfection of certain stem cell-associated genes into non-pluripotent cells, such as adult fibroblasts. Transfection is typically achieved through viral vectors, such as retroviruses. Transfected genes include the master transcriptional regulators Oct-3/4 (Pouf51) and Sox2, although it is suggested that other genes enhance the efficiency of induction. After 3-4 weeks, small numbers of transfected cells begin to become morphologically and biochemically similar to pluripotent stem cells, and are typically isolated through morphological selection, doubling time, or through a reporter gene and antibiotic selection. As used herein, iPSCs include but are not limited to first generation iPSCs, second generation iPSCs in mice, and human induced pluripotent stem cells. In some embodiments, a retroviral system is used to transform human fibroblasts into pluripotent stem cells using four pivotal genes: Oct3/4, Sox2, Klf4, and c-Myc. In alternative embodiments, a lentiviral system is used to transform somatic cells with OCT4, SOX2, NANOG, and LIN28. Genes whose expression are induced in iPSCs include but are not limited to Oct-3/4 (e.g., Pou5f1); certain members of the Sox gene family (e.g., Sox1, Sox2, Sox3, and Sox15); certain members of the Klf family (e.g., Klf1, Klf2, Klf4, and Klf5), certain members of the Myc family (e.g., C-myc, L-myc, and N-myc), Nanog, and LIN28.

More details on induced pluripotent stem cells can be found in, for example, Kaji et al., 2009, Nature 458:771-775; Woltjen et al., 2009, Nature 458:766-770; Okita et al., 2008, Science 322 (5903): 949-953; Stadtfeld et al., 2008, Science 322 (5903): 945-949; and Zhou et al., 2009, Cell Stem Cell 4 (5): 381-384.

In some aspects, examples of iPS cell lines include but not limited to iPS-DF19-9; iPS-DF19-9; iPS-DF4-3; iPS-DF6-9; iPS (Foreskin); iPS (IMR90); and iPS (IMR90). In some aspects, the stem cells are “hiPSCs,” which refers to human induced pluripotent stem cells with similar phenotypical and genotypical characteristics as human embryonic stem cells (hESCs) or human pluripotent stem cells (HPSCs). HiPSCs have self-renewing capabilities similar to hESCs and can undergo three germ layers, producing all the germ layer cells with appropriate growth factors. hiPSC organoids are organoids derived from hiPSCs.

In some aspects, compositions comprising apical-out organoids generated through any of the described methods are provided.

In some aspects, apical-out organoids generated through any of the described methods are provided.

In some aspects, compositions comprising apical-in organoids generated through any of the described methods are provided.

In some aspects, apical-in organoids generated through any of the described methods are provided.

Apical-out organoids of the invention an also be used for culturing of a pathogen and thus can be used as ex vivo infection models. Examples of pathogens that may be cultured using an apical-out organoid of the invention include viruses, bacteria, prions or fungi that cause disease in its animal host. Thus, an apical-out organoid of the invention can be used as a disease model that represents an infected state. In some aspects, the apical-out organoids can be used in vaccine development and/or production.

Diseases that can be studied by the apical-out organoids of the invention thus include genetic diseases, metabolic diseases, pathogenic diseases, inflammatory diseases etc of the intestine and/or related to intestinal development.

The apical-out organoids of the invention can be frozen and thawed and put into culture without losing their genetic integrity or phenotypic characteristics and without loss of proliferative capacity. Thus, the apical-out organoids can be easily stored and transported. Thus, in some aspects, the invention provides a frozen apical-out organoid.

For these reasons the apical-out organoids or expanded populations of cells of the invention can be a tool for drug screening, target validation, target discovery, toxicology and toxicology screens and personalized medicine.

Accordingly, in a further aspect, the invention provides the use of an apical-out organoid or cell derived from said apical-out organoid according to the invention in a drug discovery screen, toxicity assay or in medicine, such as regenerative medicine. For example, the apical-out organoid tissue may be used in a drug discovery screen, toxicity assay or in medicine, such as regenerative medicine.

For preferably high-throughput purposes, said apical-out organoids are cultured in multiwell plates such as, for example, 96 well plates or 384 well plates. Libraries of molecules are used to identify a molecule that affects said apical-out organoids. Preferred libraries comprise antibody fragment libraries, peptide phage display libraries, peptide libraries, lipid libraries, synthetic compound libraries or natural compound libraries. Furthermore, genetic libraries can be used that induce or repress the expression of one of more genes in the progeny of the stem cells. These genetic libraries comprise cDNA libraries, antisense libraries, and siRNA or other non-coding RNA libraries. The cells are preferably exposed to multiple concentrations of a test agent for a certain period of time. At the end of the exposure period, the cultures are evaluated. The term “affecting” is used to cover any change in a cell, including, but not limited to, a reduction in, or loss of, proliferation, a morphological change, and cell death.

In some embodiments, the apical-out organoids of the invention can be used to test libraries of chemicals, antibodies, natural product (plant extracts), etc for suitability for use as drugs, cosmetics and/or preventative medicines.

The invention provides the use of apical-out organoid tissue in regenerative medicine and/or transplantation. The invention also provides methods of treatment wherein the method comprises transplanting an apical-out organoid into an animal or human.

### Examples

The following examples are illustrative, but not limiting, of the compounds, compositions, and methods of the present invention. Other suitable modifications and adaptations of the variety of conditions and parameters normally encountered in clinical therapy and which are obvious to those skilled in the art are within the spirit and scope of the invention. As used herein, the use of personal pronouns such as “we”, “I”, and/or “our” refers to the inventors.

### Example 1

This example demonstrates that LPA causes apical-out polarity in ECM-free brain organoids.

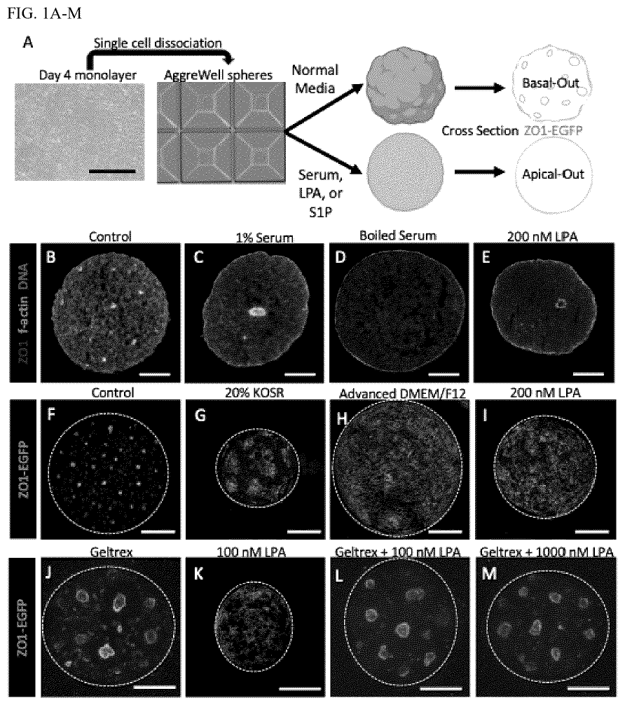

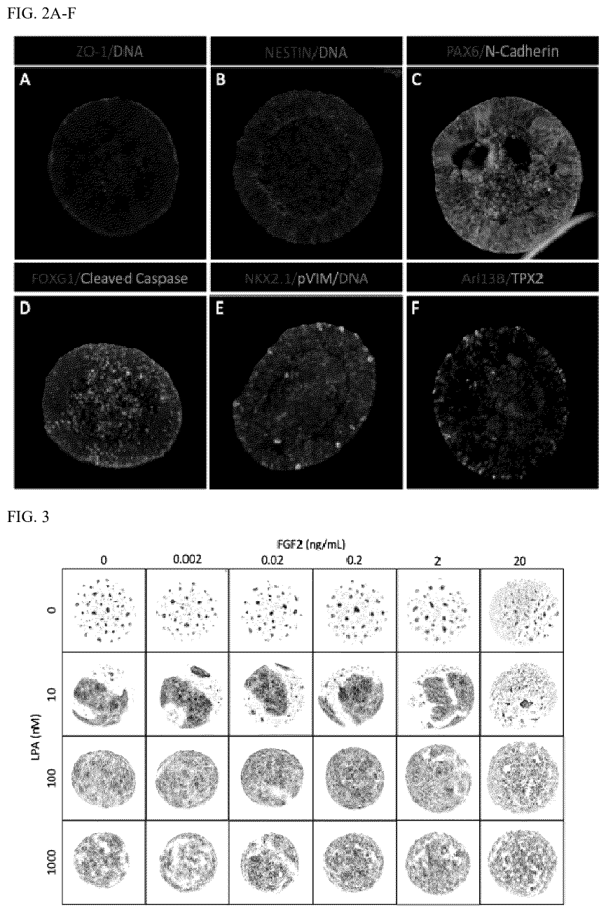

To investigate a hypothetical apical cue, we needed to first design an ECM-free brain organoid paradigm. To this end, we adapted our previously published SOSR-CO protocol by generating single cell suspensions of hiPSC-derived neuroepithelial monolayers at day 4 (when we typically start 3D conversion by placing monolayer fragments on the ECM-containing hydrogel, Geltrex) and aggregating the cells into spheres using the Aggrewell system⁶. Like other brain organoid models, these neuroepithelial spheres developed multiple rosettes with apical-in lumens within 48 hours, as shown by f-actin and ZO1 staining (FIG. 1A,B). While all current published techniques generate apical-in brain organoids, some protocols clearly displayed a transitory apical-out state when knockout serum replacement (KOSR) was present in the media⁷⁻⁹. Furthermore, experiments that showed cell-line based cysts formed apical-out after ECM or beta1-integrin removal all used serum-containing culture media, unlike brain organoid media that is serum-free^{4,5}. Therefore, we suspected that serum and KOSR may contain an apical cue. Adding 1% serum to the culture media caused robust apical-out orientation in every organoid we assessed as shown by ZO1 and f-actin on the outside edge of the organoids (FIG. 1C, FIG. 2A). In addition, the organoids continued to express neural tube markers NESTIN, PAX6, N-cadherin, and the forebrain marker FOXG1 (FIG. 2B-D). Dividing cells were also seen almost exclusively on the outside edge as shown by phosphorylated vimentin (S55) and TPX2 suggesting interkinetic nuclear migration takes place at the apical surface, now on the outside of the organoid (FIG. 2E,F).

We next sought to identify the factor in serum that induces apical-out orientation. In addition to serum, KOSR or Advanced DMEM/F12 containing media are also used in published apical-out organoid protocols^{1,2,8,9}. Both products contain AlbuMAX, a lipid rich serum albumin product. To test whether lipids may be involved in the apical-out orientation, we boiled the serum at 98° C. for 10 minutes to denature proteins. This failed to block the apical-out serum effect (FIG. 1D), suggesting that a bio-active lipid may be the apical cue. In a previous study using 2-dimensional neural rosette culture, lysophosphatidic acid (LPA) was found to have a dramatic effect on rosette structure and formation¹⁰. Therefore, we tested the effect of LPA on ECM-free brain organoids and found that 200 nM was sufficient to induce the apical-out orientation (FIG. 1E). This concentration is very close to the 190±77 nM concentration found in human CSF¹¹. The lipid concentrations in Albumax are not available from the manufacturer, but a recent publication found the total LPA concentration from Albumax I in 20% KOSR-containing media to be ˜170 nM¹². Advanced DMEM/F12 contains 400 mg/L of Albumax II, which also contains LPA but at an unknown concentration. To confirm our hypothesis that these medias resulted in the apical-out orientation observed in previous studies, we replaced our typical (control) media, DMEM/F12, with Advanced DMEM/F12 or added 20% KOSR. At this point, we began using a commercially available iPSC line with an EGFP fused to one allele of the endogenous zona occludens 1 (ZO1) gene to quickly indicate the position of the apical domain. In each case, the organoids were apical-out, similar to the 1% serum and 200 nM LPA (FIG. 1F-I). To investigate the effect of LPA when ECM is present, we added the spheres to wells coated with 100% Geltrex with or without 100 nM LPA. In each instance, when Geltrex was present (FIG. 1J-M), the organoids formed apical-in with larger lumens than without ECM (FIG. 1F), showing that the effects of ECM on orientation are greater than LPA.

A published spinal cord organoid system found apical-out orientation was dependent on FGF2 addition to the media¹³. While our organoid system does not use FGF, we tested whether its addition could have a similar effect as LPA or alter the LPA-dependent polarization in any way. At concentrations up to 2 ng/ml of FGF2, we saw no apparent effect on polarity with or without LPA. At 20 ng/mL, FGF2 still did not impact polarity but decreased the homogeneity of the ZO1-labelled tight junction barrier (FIG. 3).

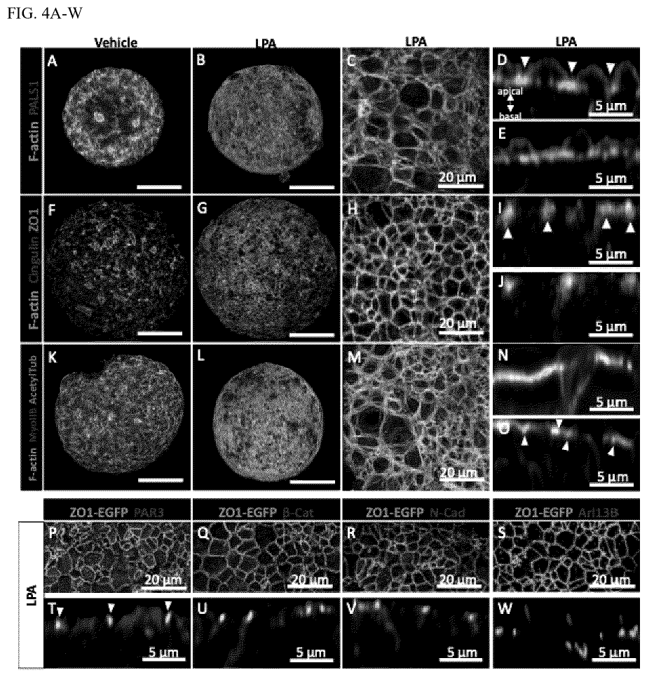

To further characterize these apical-out brain organoids, we immunostained for several important apical and structural markers. The stereotypical apical marker PALS1 (part of the Crumbs complex), tight junction markers ZO1 and cingulin were localized to internal rosettes with vehicle treatment and on the outside surface with LPA exposure (FIG. 4A-J). Myosin-IIb also became localized to the apical surface and colocalized with f-actin (FIG. 4K-O). Acetylated-tubulin showed a dramatic change from random orientation in vehicle-treated organoids (FIG. 4K) to forming rings of microtubules perpendicular to the apical membrane (FIG. 4M-O). High magnification confocal microscopy showed strong colocalization of f-actin with both ZO1 and MyoIIB (FIG. 4G,I). The important apical protein, PAR3 localizes to apical domains and ZO1-EGFP labelled tight junctions (FIG. 4P,T). Adherens junction marker proteins N-cadherin and β-catenin appear immediately basal to the tight junctions (FIG. 4Q,R,U,V) while Arl13B labelled primary cilia project above the apical surface (FIG. 4S,W). All of these proteins in the LPA-treated organoids are in the correct location for a mature apicobasally polarized cortical neuroepithelium with an outward facing apical membrane.

### Example 2

This example demonstrates that LPA-dependent orientation is specific to LPA/S1P and reversible at CSF concentrations.

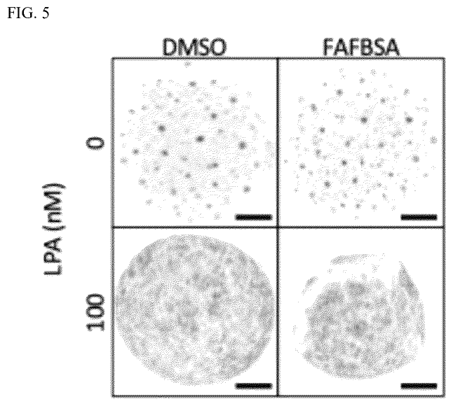

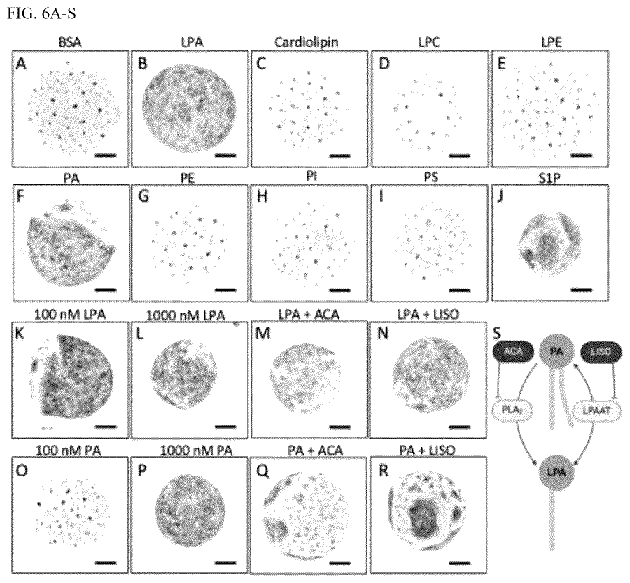

Many phospholipids are present in serum besides LPA. We sought to determine if any of these other phospholipids also induce apical-out orientation. We tested 48-hour exposure to 1 μM concentration of the following phospholipids: cardiolipin, lysophosphatidylcholine (LPC), lysophosphatidylethanolamine (LPE), phosphatidic acid (PA), phosphatidylethanolamine (PE), phosphatidylinositol (PI), phosphatidylserine (PS), and sphingosine-1-phosphate (S1P). Our initial experiments (FIG. 1) utilized LPA dissolved in DMSO; however, these other phospholipids required fatty acid-free bovine serum albumin (BSA) to be dissolved in aqueous solution. We, therefore, first compared LPA dissolved in DMSO versus BSA and found no difference in the apical-out orientation (FIG. 5). For the other phospholipids, only PA and SIP caused the apical-out orientation (FIG. 6A-J). When we compared dose-responses of PA and LPA, we found 100 nM LPA or S1P was sufficient to cause apical-out orientation, while 1000 nM, but not 100 nM, was effective for PA (FIG. 6K,L,O,P). From these data we hypothesized that PA was being converted into LPA. This conversion is achieved by phospholipase A2 (PLA2), a family of enzymes that cleave off one fatty acid chain from phospholipids (FIG. 6S). To test this idea, we added the cell-permeable PLA2 inhibitor N-(p-amylcinnamoyl) anthranilic acid, known as ACA, to the organoid media. ACA partially blocked the apical-out orientation in PA-treated organoids but not in LPA-treated organoids (FIG. 6M,Q). This finding supports our hypothesis that PA is being converted into LPA. Interestingly, lisofylline, an inhibitor of LPAAT—the enzyme that converts LPA into PA—also partially blocked the PA-dependent phenotype (FIG. 6N,R).

This result fits with the published finding that LPAAT can work in the reverse direction, converting PA into LPA14. Therefore, PA appears to cause apical-out formation through conversion to LPA. On the other hand, S1P is an independent effector of apical-out polarity as it cannot be converted into LPA (and vice versa).

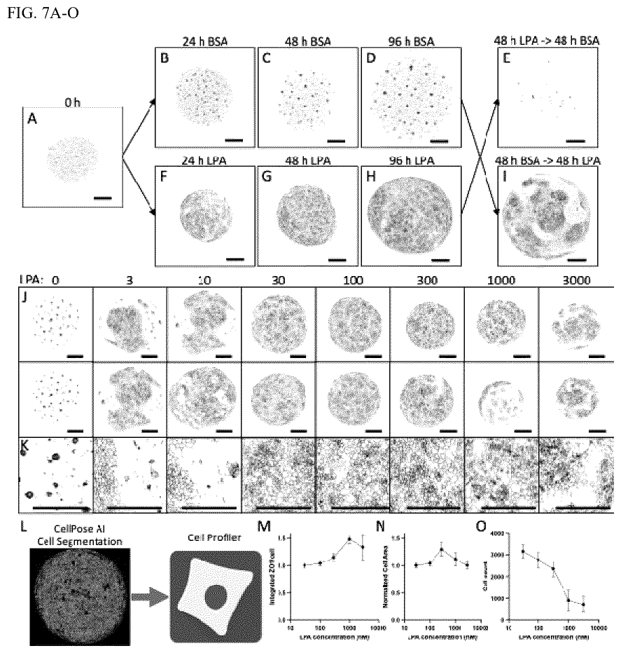

We next sought to understand whether the apical-in and apical-out phenotypes are reversible by performing a crossover experiment. To do this, we first demonstrated that LPA and BSA (vehicle) treated organoids develop their polarity within 24 hours and maintain their morphology for at least 96-hours (FIG. 7A-I). For some organoids, we instead switched culture conditions after 48 hours (BSA à LPA or LPA à BSA; FIG. 7E,I). In each case, we found the organoids had their stereotypical structure determined by their final media, indicating the LPA must be present for continued apical-out orientation and can reorient apical-in organoids when ECM is absent (FIG. 7E,I). The weakly ZO1-EGFP+puncta seen at the 0-hour timepoint appear to be primordial adherens junctions (aPJs) known to contain ZO1 and be necessary for tight junction formation (FIG. 7A)¹⁵. These puncta slowly disappear in the BSA treated organoids as small regular lumens form (FIG. 7B-D), while LPA treatment does not have these structures even at 24 hours but instead has a homogeneous tight junction network on the outer surface of the organoid (FIG. 7F).

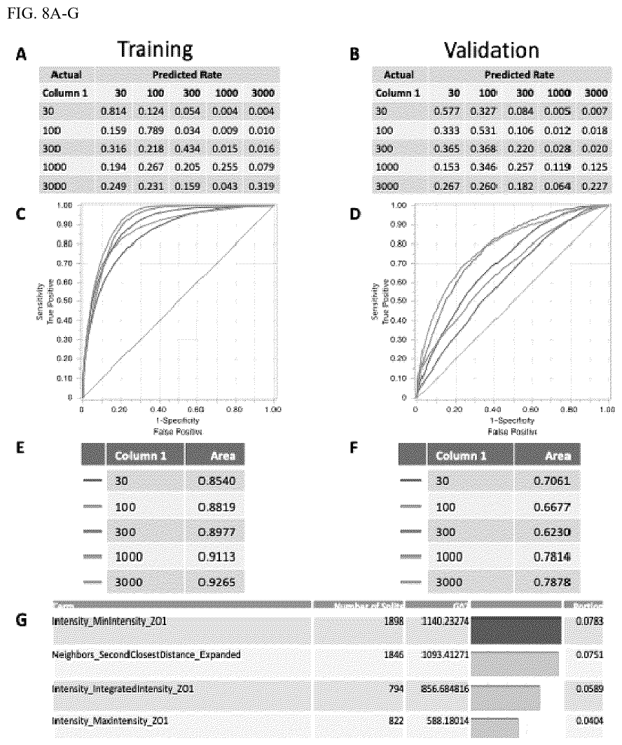

We also measured the relationship between LPA concentration and apical-out formation. For 3 and 10 nM LPA, we found an intermediate phenotype with apical-out and apical-in regions (FIG. 7J). For 30-300 nM, we saw nearly ideal apical-out ZO1-EGFP while 1000 and 3000 nM had regions devoid of either apical-in or apical-out ZO1, indicating a loss of tight-junction barrier (FIG. 7K). The optimal concentration range (30-300 nM) fits with the 190±77 nM LPA concentration found in adult human CSF¹¹. To further investigate the dose-dependent effects of LPA, we performed cell segmentation of individual apical cell surfaces using CellPose AI software which identified >40,000 cells in 22 organoids. These segmented cells were than analyzed for shape, size, and fluorescent properties using Cell Profiler. These data were limited to the 30-3000 nM range with consistent apical-out orientation since the apical-in rosettes would yield drastically different metrics. We trained a boot-strap random forest model to this data set for the prediction of the LPA concentration with a high predictive value in the validation set ROC curve (FIG. 8). One of the most informative metrics for the model was the integrated ZO1 fluorescence (FIG. 7M), which increased with LPA concentration over the 30-1000 nM range. We explored whether this increase resulted from changes in cell size. Although 300 nM LPA exposure increased cell size, this effect did not account for the ZO1 fluorescence trend (FIG. 7N), indicating that LPA causes a dose-dependent increase in ZO1 expression. Conversely, high concentrations of LPA led to regions that were ZO1 negative resulting in a smaller number of segmented cells in each organoid (FIG. 7K,O).

### Example 3

This example demonstrates that LPA and S1P signaling through GPCR/RhoA/ROCK causes apical out polarity.

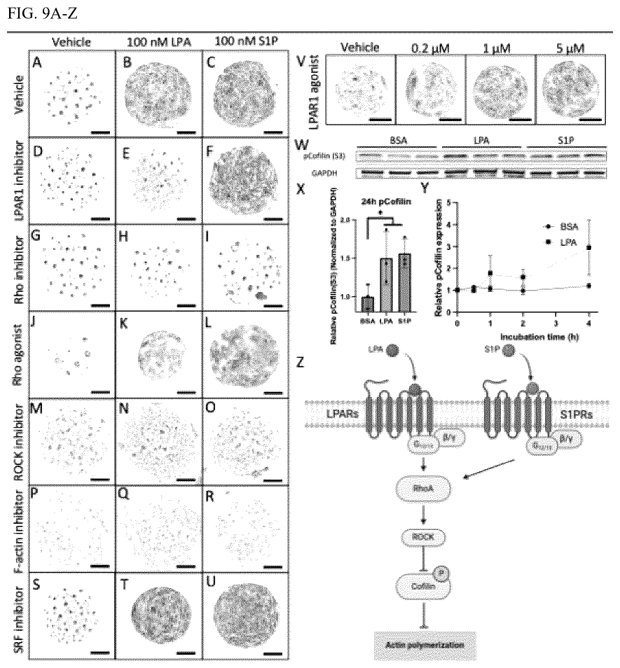

Given the specificity of LPA and S1P, we hypothesized that the apical-out polarity is likely due to GPCR signaling through several characterized LPA and SIP GPCRs. LPA has at least 6 GPCRs (LPAR1-6) and S1P has 5 (S1PR1-5), and all are involved in various canonical g-protein activated pathways, including G12/13/RhoA/ROCK, GqPLC, Gi/Ras/MAPK, Gi/PI3K, and GS/cAMP. LPAR1 is highly expressed in the neural tube and developing cortex, and LPAR1 knockout mice have exencephaly or craniofacial defects¹⁶. Therefore, we tested the effect of the LPAR1/3 inhibitor KI16425. At an optimal LPA concentration (100 nM) to induce apical-out organoids, we found that 20 μM KI16425 completely blocks apical-out induction but had no effect on S1P-treated (100 nM) organoids as expected (FIG. 9A-F). From our previous RNA sequencing on day 6 organoids, we found high expression of LPAR1, LPAR2, and LPAR4 mRNA¹⁷. Since this inhibitor only affects LPAR1 and LPAR3, LPA appears to affect polarity only through LPAR1 in our brain organoid system. These data also indicate that S1P does not function via LPAR1, but we did not characterize the specific receptor as SIP is not expressed in CSF^{18,19}. We further tested whether LPAR1 agonism alone could cause apical-out orientation using the LPAR1-specific agonist UCM-05194. We observed a partial effect of UCM-05194 at 0.2 μM, which is near the known EC50 (0.24 μM), and a more complete induction of apical-out organoids at 1 μM and 5 μM concentrations (FIG. 9V).

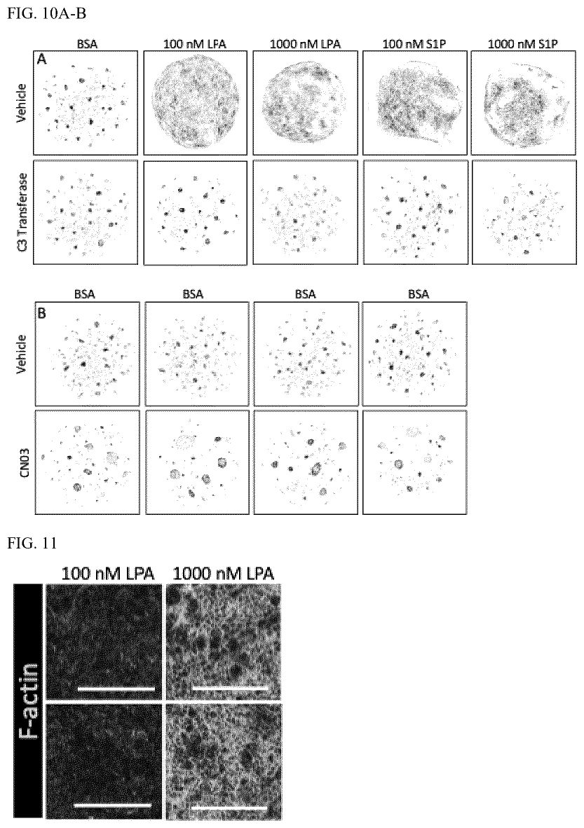

Based on these data and previous reports that showed G12/G13 expression were necessary for neural tube formation and closure²⁰, we hypothesized that LPA and S1P induce apical-out orientation via the G12/13/RhoA/ROCK/Cofilin pathway downstream of their GPCRs. To test this hypothesis, we exposed organoids to LPA or S1P along with the Rho inhibitor C3 transferase. For either lipid at 100 nM, the Rho inhibitor, C3-transferase completely blocked the formation of apical-out organoids (FIG. 9G-I). The inhibitor was also effective when higher concentrations (1000 nM) of LPA or S1P were used (FIG. 10). Treatment with the cell-permeable Rho activator, CN03 had no effect on LPA or S1P-dependent apical orientation (FIG. 9K,L); however, in the vehicle condition (BSA), the RhoA activator drastically altered the number, size, and shape of apical-in rosettes (FIG. 9J and FIG. 10B). The number of rosettes was reduced, the size was dramatically increased, and the shapes were more spherical, appearing like the organoids treated with ECM (FIG. 1J). These data indicate that Rho signaling is necessary, for apical-out formation, and suggest that LPA and S1P provide a gradient from the outside of the organoid resulting in the apical-out orientation while the cell permeable activator CN03 does not. These data indicate that either ECM or LPA can activate Rho but with differing effects on polarity.

We next tested the Rho-kinase (ROCK) inhibitor, Y27632, and found that it completely blocked the apical-out inducing effects of both 100 nM LPA and SIP, suggesting that RhoA activates downstream ROCK signaling during apical-out organoid induction (FIG. 9M-O). The ROCK inhibitor caused complete loss of apical-out ZO1 or apical-in ZO1 rosette formation. The images look like the 0-hour timepoint in FIG. 7A, indicating that ROCK likely plays a role in both apical-out and apical-in orientation of tight junctions. However, since RhoA inhibition does not impair apical-in rosette formation ROCK function does not seem to be due to RhoA activation in the vehicle treatment condition. ROCK regulates actin polymerization through several effectors including inactivation of cofilin and activation of mDia1. Cofilin is an enzyme that severs filamentous actin (F-actin), except when it is phosphorylated at serine 3. This is achieved by LIMK1/2 when phosphorylated by ROCK1/2. Therefore, we measured p (S3)-Cofilin after LPA or SIP exposure (1000 nM) and found a robust increase in phosphorylation at 24-hours for both lipids (FIG. 9W-Y). In a time-series using LPA, we observed that this increase began after 1 hour of exposure with no change in phosphorylation in the vehicle condition (FIG. 9Y). To directly test whether actin polymerization is important for apical constriction, we inhibited actin polymerization using cytochalasin B. The results were similar to ROCK inhibition with many ZO1-EGFP puncta throughout, indicating a complete lack of polarization (FIG. 9P-R). As further evidence that LPA affects f-actin polymerization, we generated apical-out organoids with either 100 or 1000 nM LPA and found a dramatic increase in f-actin from the 1000 nM LPA treatment as seen by wholemount phalloidin staining (FIG. 11). Taken together, these data indicate that LPA/S1P induces apical-out orientation via a pathway involving GPCR/RhoA/ROCK/LIMK/cofilin/Factin.

Rho activation and f-actin polymerization are also known to cause the serum response factor (SRF) to translocate to the nucleus and induce transcription of genes with serum response elements (SREs). To test whether this downstream transcription is necessary for LPA-induced apical-out orientation, we treated with the SRF inhibitor, CCG-1423 (10 μM). This inhibitor had no discernable effect on either the apical-in (BSA) or apical-out (LPA) orientation or morphology, indicating that SRF-dependent transcription does not play a role in initial polarization (FIG. 9S-U).

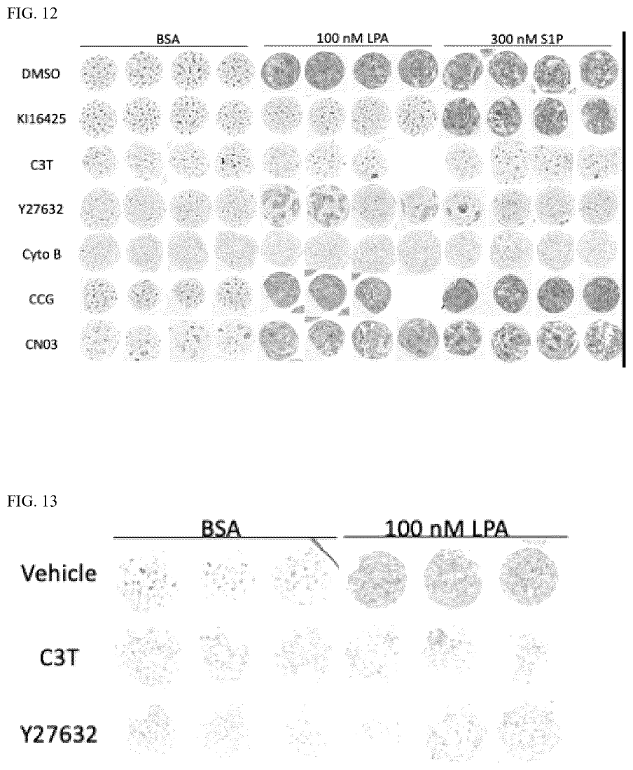

Replicate organoids for each of these conditions are presented in FIG. 12 for the ZO1-EGFP expressing line do demonstrate that our phenotypes are robust. Since this line was from a Japanese male donor, we tested the reproducibility of the LPA phenotype on organoids from a control iPSC line RPChiPS8023G1 that is from a Hispanic female donor. This line did not have the ZO1-EGFP fusion protein; therefore, we immunostained for ZO1. We found that this line also becomes apical out with 100 nM LPA and the effect is blocked by the Rho inhibitor (C3-transferase) and ROCK inhibitor (Y27632) (FIG. 13).

### Example 4

This example demonstrates that LPA maintains apical-out orientation for over one-month with increased numbers and enhanced morphology of radial glia.

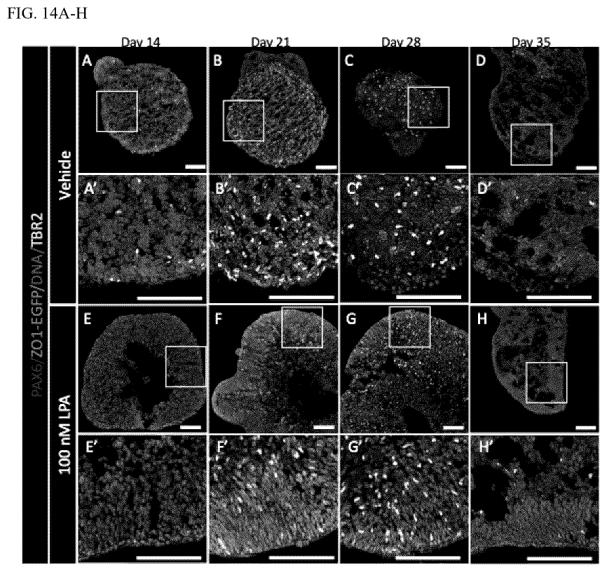

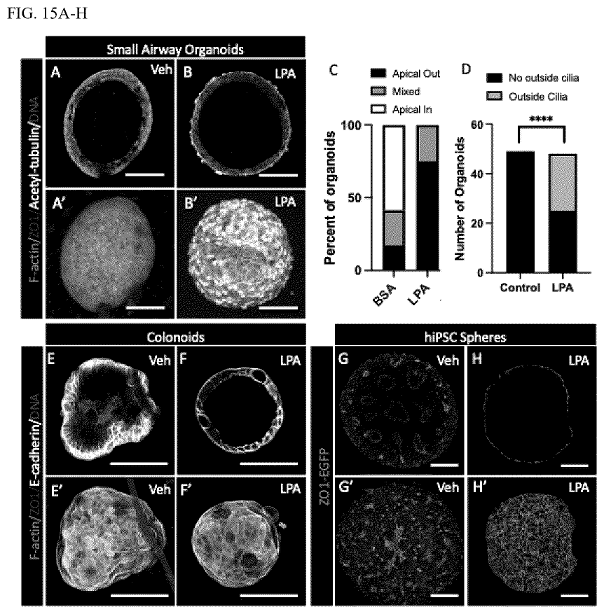

FIG. 14 demonstrates that long-term LPA causes persistent apical-out orientation and growth factor like effects. A-H′ Organoids were treated with vehicle (A-D′) or 100 nM LPA (E-H′) starting on day 5. Organoids were cryosectioned and immunostained on for PAX6 (red), TBR2 (white), ZO1-EGFP (green), and DNA (blue). Whole section images (A-H) and magnified insets (A′-H′). Scale bars are 100 μm.

To test whether the effects of LPA were transitory, we exposed brain organoids to 100 nM LPA continuously starting on day 5 and fixed samples each week for 5 weeks. Because these organoids were too large for whole mount staining, we employed cryo-sectioning prior to immunostaining. At each timepoint, the polarity remained apical-out as shown by ZO1-EGFP on the outside edge while those without LPA had a few small central lumens (FIG. 14). PAX6, a marker of forebrain radial glia, labelled most cells at each condition and timepoint. However, the LPA-treated organoids had more cells expressing PAX6, more consistent fluorescent intensity across PAX6-expressing cells, and a more radial orientation closely resembling a pseudostratified neuroepithelial-like morphology. Furthermore, LPA exposed organoids had a reduced number of condensed nuclei with bright DAPI signal. Notably, mice with LPA injected in the ventricles had elevated numbers of TBR2 positive intermediate progenitors in the developing cortex²¹. While we did not see an obvious increase in the number of TBR2+ cells, we did observe a possible shift towards later generation of TBR2-expressing cells in the organoids with LPA treatment (FIG. 14).

We identified a large necrotic core in the center of each apical-out organoid at these later timepoints. From 48-hour LPA organoid cryo-sections, we see a pseudostratified neuroepithelial-like layer that extends 1/3 to ½ of the radius of the organoids as shown by nestin or N-Cadherin staining (FIG. 2B,C). While still expressing the markers of the anterior neural tube (FOXG1, PAX6, nestin, N-cadherin), the center cells do not appear to connect to this layer or extend processes to the organoid surface. This core is strongly caspase-3 positive (FIG. 2D) likely due to isolation from media nutrients and oxygen due to the complete barrier formed by the tight junctions surrounding the surface of the organoid (FIG. 2A). We hypothesize that apical-out barrier formation could be the cause for the necrotic cores seen in brain organoid techniques that use KOSR-based media. These systems form temporary apical-out brain organoids (until KOSR removal) as shown by atypical PKC and N-cadherin in these publications^{8,9}.

### Example 5

This example demonstrates that LPA orients intestinal and lung organoids.

Up to this point, our data indicate the role of LPA in apicobasal polarization in the neuroepithelium. We next asked whether LPA serves as a critical apical cue for other organoid systems. To investigate this possibility, we tested the effects of LPA on patient derived lung and colon epithelial organoids, spheres of undifferentiated hiPSCs, and iPSC-derived multilineage intestinal organoids. Lung organoids consisting of epithelium from the small airways derived from adult patient donor lungs were expanded and removed from Matrigel according to Co et al 2021¹. Lung organoids were resuspended in growth medium using DMEM/F12 with or without 100 nM LPA. We selected apical-in polarized lung organoids with an obvious single lumen into 96-well low adherence dishes. Those treated with LPA also had a greater number of apical out organoids as shown by the position of the ZO1, f-actin, and acetyl-tubulin labelled cilia. (FIG. 15A-B′). After 5 days of exposure prior to fixation and immunostaining, motile cilia could easily be observed by light microscopy using phase contrast in all organoids. The number of organoids with cilia on the outer surface were counted. Only organoids exposed to LPA had any exterior cilia (23/48 with LPA, 0/49 in control media) (FIG. 15A-B′ quantified in FIG. 15D). It should be noted that apical-in lumens had motile cilia observable by phase microscopy that did not label with the acetyl-tubulin antibody (FIG. 7A-B′).

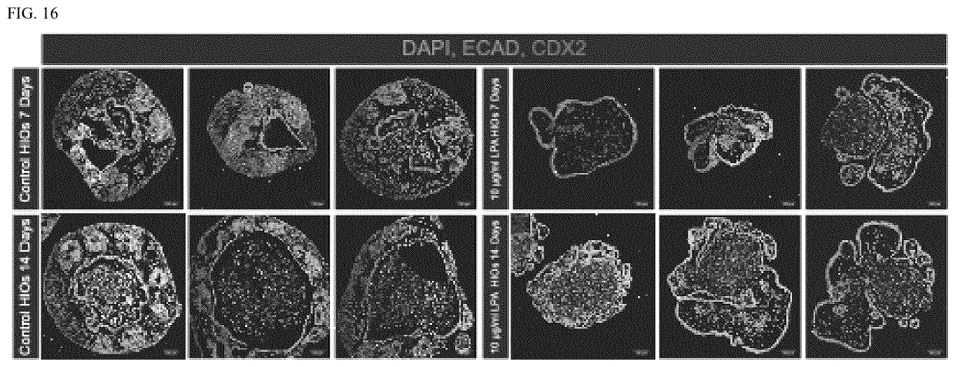

Patient derived colon organoids (also called colonoids) were also generated and removed from Matrigel before being suspended in media alone or with 100 nM LPA. We found colonoids in media were mostly mixed polarity with ZO1 and F-actin on the outside surface and on internal lumens. This results from having a bilayer of cells that yield dysmorphic, “bumpy” organoid shapes (FIG. 15E-E′). LPA-treated colonoids had spherical organoids that were a single cell layer thick (FIG. 15F). ZO1 and f-actin label the outside of the organoids with E-cadherin labeled adherens junctions perpendicular to the outside apical surface. The LPA-treated organoids also had stronger ZO1 fluorescent signal and had bulging cells indicative of goblet cells at a timepoint that typically does not have this level of maturity (FIG. 15F′). Previous studies including from our group of generated multilineage human intestinal organoids from induced pluripotent stem cells^{22,23}. These organoids have epithelial, mesenchymal, and even endothelial cell types with vascularized structure. Using the aggregation technique with hindgut endoderm, we skipped the embedding step into Matrigel domes and instead tested the effects of LPA on suspended organoids. We then immunostained sections for the epithelial markers E-Cadherin and CDX2. Similarly to previous publications, the suspended organoids lacking LPA had apical-in epithelial structures encase in mesenchymal and endothelial cells. Treatment with LPA caused the inverse in every organoid we imaged: apical-out epithelium on the outer edge of the organoid encasing the other cell types (FIG. 16). Thus, LPA can induce apical-out organization of epithelial cells even in a multilineage organoid context.

Because human pluripotent stem cells form epithelial-like colonies in their primed state, we tested whether LPA could cause apical-out formation in hiPSC spheres. Using the same paradigm as the neuroepithelial cell spheres, we placed Aggrewell generated hiPSC spheres in mTeSR-Plus media alone or with 100 nM LPA added. All organoids treated with LPA formed ZO1-EGFP labeled apical-out organoids while mTeSR-plus alone resulted in apical-in lumens (FIG. 15G-H′). These data demonstrate that LPA is a broad apical cue in the absence of ECM for multiple cell lineages and developmental timepoints, and that LPA is likely the cause of many-if not all-apical-out organoid systems.

### Example 6

This example provides a discussion related to Examples 1-5.

In the experiments described in Examples 1-5, we show that apical-out orientation in ECM-free brain organoid culture is induced by two phospholipids found in serum: LPA and S1P. These phospholipids act as exogenous apical cues that can be used to orient apicobasal polarity in the absence of extracellular matrix signaling, and they function by activating their respective GPCRs. We also find that this pathway involves the downstream activation of Rho, which is necessary, for apical-out brain organoid orientation. Notably, the different culture media that we have identified in the literature as producing apical-out organoids, including for intestinal and lung organoids, contain LPA1-5,24,25. Given that both phospholipids are present in amniotic fluid but only LPA is found in CSF at concentrations that induced apical-out organoids in our assay, we propose that both LPA and SIP are critical for the induction of neuroepithelial polarity in the neural plate/tube, and that LPA in CSF is necessary for maintaining polarity and barrier function at the ventricles.

Previous work has shown that apical-out inversion in MDCK cells induced by incubation with beta1-integrin blocking antibody is RhoA dependent²⁴. This inversion could also be produced by hanging-droplet suspension²⁵. Furthermore, apical-out polarity inversion of suspension micropapillary carcinoma cells is known to be Rho/ROCK dependent²⁶. In each case, the cells were grown in suspension in media containing serum; thus, LPA and S1P could direct the apical-out orientation. The use of LPA- or S1P-containing media under basal conditions for most cell culture systems (either in serum, knockout serum replacement, or Albumax containing medias like Advanced DMEM/F12) is likely the reason that LPA and S1P have not been previously identified as apical cues. Therefore, our study connects the known RhoA/ROCK pathway activation necessary for apical-out formation to LPA and S1P found in these medias.

In the context of neural tube formation and closure, apicobasal polarity is necessary for proper formation since the rounded tube forms due to polarized constriction of the apical surface of each cell. Knockout of each part of the pathway, including the LPA-producing enzyme (autotaxin/ENPP2), LPA receptor (LPAR1), G13 (GNA13), RhoA, or pharmacological inhibition of ROCK1/2 can lead to loss of neural tube closure and/or exencephaly ^{16,27-30} LPA is expressed between 100-200 nM in CSF (and serum) where it is produced in the choroid plexus by autotaxin¹¹. This LPA concentration was optimal in our brain organoid system to induce apical-out polarity. On the other hand, S1P is exceptionally low in CSF (1-2 nM) and high in serum (400-1000 nM) 19,31. Therefore, it is surprising that loss of S1P production in mice from SphK1 and SphK2 knockout also resulted in lack of neural tube closure despite low S1P in CSF³². The concentration of LPA and SIP in amniotic fluid are ˜27 and 20 nM, respectively^{33,34}, and based on our apical-out induction with 30 nM LPA, this should be sufficient to provide the apical cue signal. Therefore, LPA and S1P in amniotic fluid may both be needed for proper neural tube closure, but only LPA is needed in CSF for proper barrier maintenance at the CSF/VZ interface. In the future, amniotic fluid should be tested for its capacity to induce apical-out organoid formation.

Interestingly, knockout of Enpp2, SphK1/SphK2, or GNA13 also leads to failure of vasculature formation, highlighting the importance of this signaling pathway for tube formation beyond the neural tube alone^{27,28,32}. Since these genes are all involved in LPA/S1P synthesis or signaling, these studies suggest that LPA and S1P are not only apical cues but also necessary for neural tube and vasculature formation, even in the presence of ECM proteins. The importance of these phospholipids seems highly conserved. For example, loss of the sphingolipid producing enzyme in C. elegans results in the formation of many circular intestinal lumens rather than one contiguous tube³⁵. Therefore, although ECM-integrin signaling is sufficient for apicobasal polarity, loss of a phospholipid apical instructive cue may lead to loss of tubulogenesis due to lack of contiguous apical membranes.

Our optimal concentration for apical-out formation of 100 nM LPA is lower than the typical concentrations used to elicit LPA-dependent phenotypes, which are typically 1-10 μM^{10,36}. Serum has ˜10 μM LPA; therefore, typical 10% serum containing media often used for cell culture would have a concentration of 1 μM LPA. Interestingly, plasma has a concentration range of 0.7-80 nM, and the elevated LPA in serum is due to the role of LPA in platelet activation³⁷. We observe disruption of the tight junction barrier formation and excessive f-actin in the high concentration range (1-10 μM). These disruptive effects of elevated LPA in our model may explain why overexpression of autotaxin leads to loss of neural tube closure and injection of excess LPA in ventricles leading to the loss of ventricular barrier integrity^{38,39}. Therefore, animal models are congruent with our dose-response data that too much or too little LPA is detrimental to the formation and stability of the neural tube.

The results of this study also have implications for cancer biology where polarity switching often occurs in metastatic cancers in the blood stream. One report describes blockade of cancer cell polarity switching by Rho/ROCK inhibitors applied in suspension culture²⁶. Given our findings, this polarity switching may be induced by apical cue phospholipids, LPA and S1P, present in blood or cell culture media. This knowledge may provide additional targets for treating metastatic cancers.

Reproducible production of apical-out brain organoids opens many future research possibilities. As we have shown, the apical surfaces can be imaged in whole mount organoids at high magnification allowing for better understanding of protein localization at the tight-junctions and apical membrane in neural tube organoids. These organoids will allow for investigation of primary cilia, growth factor receptors, and nutrient absorption that occur from CSF to the apical membranes of neuroepithelial cells, followed by those of radial glial and ependymal cells that line the brain ventricles at different stages of development. These organoids will also provide useful models for studying viral or bacterial infections that can occur in the CSF by providing direct access to the interfacing surface.

### Example 7

This example describes the materials and methods implemented in the experiments described in Examples 1-6.

iPSC Lines

We purchased the AICS-0023 cell line from the Coriell Institute Biorepository (Camden, NJ, USA) in the Allen Cell collection. This line contains a monoallelic mEGFP-TJP1 (which encodes for the zona occludens-1 [ZO1] protein) to label the lumen in live SOSR-COs. We have previously published a control iPSC line generated from commercially available foreskin fibroblasts that we used for experiments utilizing Phalloidin-Alexa488 to measure f-actin¹⁷. Key phospholipid and inhibitor data was confirmed in the commercially available control iPSC line RPChiPS8023G1 from ReproCell.

iPSC Culture

The iPSC cultures were maintained on 6-well TC dishes coated with Geltrex (Thermo, Waltham, Ma, USA) diluted 1:200 dilution in DMEM/F12 medium (Thermo) at 37° C. Cells were cultured in mTeSR1 or mTeSR-Plus medium (Stemcell Technologies, Vancouver, BC, Canada). When the colonies reached ˜40% confluency, the cultures were incubated with 1 ml of hypertonic solution containing sodium citrate and potassium chloride for 2 min¹⁷. The solution was then replaced with culture medium, and the colonies were mechanically detached with a mini cell scraper. The solution was then pipetted up and down 3-6 times to break the colonies into smaller pieces. The colony fragment suspension was then re-plated at a dilution of 1:8 onto newly Geltrex-coated plates.

### ECM-Free Organoid Differentiation

We Accutase passaged 1×10⁶ iPSCs onto Geltrex-coated (1:50 dilution in DMEM/F12) 12-well plates in mTeSR with 10 μM Y-27632 (Tocris, Bristol, UK). When the cells reached 80-100% confluency, the medium was changed to 3N (50:50 DMEM/F12: neurobasal with N₂ and B27 supplements [Thermo]) without vitamin A with 2 μM DMH-1 (Tocris), 2 μM XAV939 (Cayman Chemical, Ann Arbor, MI, USA), and 10 μM SB431542 (Cayman Chemical) with 2 mL of medium per well. Subsequently, 1.5 mL medium were changed daily with 1 μM cyclopamine (Cayman Chemical) beginning on day 1 along with the other 3 inhibitors. On day 4, the monolayer was dissociated by incubating with Accutase for 8 minutes. After centrifugation, the cells were resuspended in media with the 4 inhibitors and 10 μM Y-27632. 2×10⁶ cells were added to each well of a 24 well Aggrewell 800 in 2 mL of media, and the plate was centrifuged at 100 g for 3 minutes. The following day, individual spheres were transferred to wells of a low-adherence U-bottom 96 well plate in 3N-A media with 3 μM CHIR99021 and test compounds such as lysophosphatidic acid.

### Immunostaining

Organoids were fixed in 4% paraformaldehyde in PBS for 1 hour at 4 C. For cryosectioning, the organoids were embedded in OCT, and 15 μm sections were attached to slides. After PBS washes to remove OCT, sections were permeabilized with 0.1% TritonX-100 for 20 minutes. Each slide incubated with ICC buffer with phosphate-buffered saline, with 5% normal goat serum, 1% BSA, and 0.05% Tween-20. Primary antibodies were diluted in ICC buffer according to the dilutions in Table SX and incubated overnight at 4 C. Each was washed 4× in PBS+0.05% Tween-20 for X min each. Secondary antibodies (either Goat anti-rabbit-Alexa568 or Goat anti-mouse-Alexa647) were diluted 1:1000 in ICC buffer and incubated for 90 min. The sections were then counterstained with Hoescht DNA dye for 10 minutes and coverslips mounted using Glycergel. For wholemount, organoids were permeabilized and blocked in ICC buffer with 0.1% TritonX-100 over night at 4° C. on a Nutating shaker. Primary antibodies were added in this same ICC buffer with TX-100 overnight at 4° C. on a Nutating shaker. The organoids were washed 4×15 minutes in PBS with 0.05% Tween-20. Secondary antibodies were added at 1:1000 dilution in ICC buffer with TX-100 at 4° C. on a Nutating shaker. After 4 additional washes in PBS with Tween-20, the organoids were placed thin bottom 96 well plates in PBS for imaging. Microscopy was performed with an Andor BC43 confocal microscope with 20× objective. Z-series of the bottom 100 μm for each organoid were obtained with a 1.5 μm step size (67 images each). Maximum projections were obtained with the same brightness and background subtraction applied for each experiment.

### Immunoblotting

Total protein was extracted using CelLytic™ M Cell Lysis Reagent (Sigma-Aldrich, #C2978) with PhosSTOP™ (Roche, #04906845001) and cOmplete™ ULTRA Protease Inhibitor Cocktail (Roche, #05892970001). Protein concentrations were determined by Qubit™ Protein BR Assay kit (Invitrogen, #A50668). Equal amounts of protein were denatured at 99° C. for 5 minutes, separated by SDS-PAGE, and transferred onto NC membranes. The blots were blocked with 5% nonfat dry milk in Tris-buffered saline with 0.1% Tween 20 (TBS-T) for 1 hour, followed by incubation with primary antibodies at 4° C. overnight (Antibodies and dilutions in Table SX). The blots were washed with TBS-T followed by incubation with IRDye 800 CW goat anti-rabbit or goat anti-mouse secondary antibodies (LI-COR Biosciences, 1:8000) for 1 hour. After additional washes with TBS-T, the immune complexes were detected by the Odyssey Imaging Systems (LI-COR Biosciences). Image Studio™ Lite Software (LI-COR Biosciences) was used to quantify the protein signals.

### Lipid Solutions

Lyophilized lipid powders were dissolved in soluble organic solvents. The solutions were aliquoted into glass culture test tubes to achieve 0.3 mg phospholipid each. The solvent was then removed using a Savant Speed Vac with solvent collection flask for 0.5-3 hours until only a phospholipid film or small particulates remained on the glass. The film was then dissolved into a 10% fatty acid free BSA solution (catalog number) with warming at 37 C for 30 minutes followed by vortexing and additional warming as needed until fully dissolves to a concentration of 0.3 mM. The solutions were then sterile filtered through a low protein binding 0.45 μm syringe filter. LPA was also dissolved in DMSO and found to have no difference in orientation compared with the BSA solution (FIG. 5).

### Patient-Derived Lung Organoid Culture and ECM Removal

HT-617 airway organoids were maintained in Matrigel with FANY media. Organoids were move to a 15 mL conical tube with 10 mL EDTA and rotated in cold room for 1 hour. The organoids were pelleted at 300 g for 3 minutes. The EDTA was aspirated and the pellet washed with 5 mL of DMEM/12. The organoids were pelleted at 300 g for 3 min. The DMEM/F12 was removed and the organoids were resuspended in FANY media with the BSA replaced with fatty acid free BSA. Organoids with a clear apical lumen were picked into a low-adherence U-bottom 96 well plate the FAFBSA containing FANY with or without 100 nM LPA. The cultures were fixed and whole mount immunostained.

### Patient-Derived Colonoid Culture and ECM Removal

Colon-88 colon organoids were maintained in Matrigel with L-WRN Conditioned Media for 6 days prior to harvesting. The organoids were transferred to a 50 ml conical with 45 mL 5 mM EDTA and rocked in a cold room for 1 hour. Organoids were pelleted at 300 g for 3 minutes. The EDTA was removed, and the pellet was washed in 15 mL DMEM/F12. Organoids were pelleted at 300 g for 3 minutes. Organoids were resuspended in 0.567 mL of DMEM/F12. 81 μL of resuspended organoid mixture was transferred to 6 wells of a low-adherence 6 well plate. The wells contained Advanced DMEM/F12, DMEM/F12, and DMEM/F12+100 nM LPA, with 2 wells per condition. After 24 hrs, one of each well was fixed and whole mount immunostained. After 48 hrs, the remaining wells were fixed and whole mount immunostained.

### Human Intestinal Organoids (HIOs) and Slide Preparation

Human Intestinal Organoids (HIOs) were generated from iPSCs (line iPSC72.3⁴²) by aggregation as previously described^{22,23}, with the exception that a 96-Well U Bottom plate was used. HIOs were either immediately grown in 10 μg/ml of LPA in addition to growth media or grown under normal growth media conditions for 10 days after seeding. Media was changed every 3-4 days. HIOs were placed in 10% Neutral Buffered Formalin at room temperature on a rocker for 24 hours to allow for complete fixation. After fixation, HIOs were washed three times in UltraPure DNase/RNase-Free Distilled Water for 1 hour each. The tissue was dehydrated through a methanol series diluted in UltraPure DNase/RNase-Free Distilled Water for 60 minutes per solution in the following order: 25% MeOH, 50% MeOH, 75% MeOH, 100% MeOH. Dehydrated tissue was placed in 100% EtOH, followed by 70% EtOH, and then perfused with paraffin using an automated tissue processor (Leica ASP300) with solution changes every hour overnight. Tissue was subsequently placed into tissue cassettes and base molds for sectioning. Sections, 5 μm-thick, were cut from paraffin blocks onto charged glass slides. Slides were dried for 1 hour at 60° C. and used within a week for immunofluorescence staining. Slides were deparaffinized with two 5-minute washes in Histo-Clear II, followed by rehydration through a graded ethanol series: two 2-minute washes each in 100% EtOH, 95% EtOH, 70% EtOH, 30% EtOH, and finally two 5-minute washes in ddH2O. Antigen retrieval was carried out in 100 mM trisodium citrate with 0.5% Tween 20 pH 6.0. Slides were placed in a pressure cooker for ˜30 minutes, cooled, and washed three times in ddH2O for 5 minutes each. Slides were subsequently immunostained as described previously.

### Quantification and Statistical Analysis

For all brain organoid, iPSC spheroid, and human intestinal organoid (HIO) data presented without quantification, the results were the same in all replicate organoids (3-6/condition) across 2+experiments. The effects of LPA on lung organoids and colonoids was variable; therefore, for the lung organoids we performed a Chi square analysis between the control and LPA-treated conditions.

Key resources table

| | | |
|---|---|---|
| REAGENT or RESOURCE | SOURCE | IDENTIFIER |

Antibodies

| | | |
|---|---|---|
| Mouse monoclonal anti-Acetyl- | Sigma | Cat#T6793; |
| tubulin | | RRID: AB_477585 |
| Mouse monoclonal anti-Arl13B | NeuroMab | Cat#N295B/66; |
| | | RRID: AB2877361 |
| Mouse monoclonal anti-β-Catenin | BD Biosciences | Cat#610153; |
| | | RRID: AB_397554 |
| Rabbit polyclonal anti-Cingulin | Invitrogen | PIPA555661 |
| Rabbit monoclonal anti-Cleaved | BD Biosciences | Cat#559565; |
| Caspase-3 | | RRID: AB_397274 |
| Mouse monoclonal anti-CDX2 | BioGenex | Cat#MU392A-UC; |
| | | RRID: AB_3101998 |
| Goat polyclonal anti-E-Cadherin | R&D Systems | Cat#AF748; |
| | | RRID: AB_355568 |
| Mouse monoclonal anti-E-Cadherin | BD Biosciences | Cat#610182; |
| | | RRID: AB_397581 |
| Mouse monoclonal anti-GAPDH | Sigma | Cat#G8795; |
| | | RRID: AB_1078991 |
| Rabbit monoclonal anti-MyoIIB | Cell Signaling | Cat#8824; |
| | | RRID: AB_11217639 |
| Rabbit polyclonal anti-Myosin IIb | Biolegend | Cat#909901; |
| | | RRID: AB_2565101 |
| Mouse monoclonal anti-N-Cadherin | Thermo Fisher | Cat#33-3900; |
| | | RRID: AB_2313779 |
| Mouse monoclonal anti-Nestin | Millipore | Cat#MAB5326; |
| | | RRID: AB_2251134 |
| Rabbit polyclonal anti-MPP5/PALS1 | Proteintech | Cat#17710-1-AP; |
| | | RRID: AB_2282012 |
| Rabbit polyclonal anti-Partitioning- | Millipore | Cat#07-330; |
| defective 3 (PAR3) | | RRID: AB_2101325 |
| Rabbit polyclonal anti-PAX6 | MBL | Cat#PD022; |
| | | RRID: AB_1520876 |
| Rabbit monoclonal anti-Phospho- | Cell Signaling | Cat#3313; |
| Cofilin-(S3) | | RRID: AB_2080597 |
| Mouse monoclonal anti-Phospho- | MBL | Cat#D076-3; |
| Vimentin (S55) | | RRID: AB_592963 |
| Mouse monoclonal anti TBR2(EOMES) | R&D | Cat#MAB6166; |
| | | RRID: AB_10919889 |
| Rabbit polyclonal anti-TPX2 | Novus | Cat#NB500-179; |
| | | AB_10002747 |
| Rabbit polyclonal anti-ZO1 | Thermo Fisher | Cat#61-7300; |
| | | RRID: AB_2533938 |
| Mouse monoclonal anti-ZO1 | Thermo Fisher | Cat#33-9100; |
| | | RRID: AB_2533147 |

Chemicals, peptides, and recombinant proteins

| | | |
|---|---|---|
| ACA | Medchem Express | Cat#HY-118628 |
| C3-Transferase | Cytoskeleton, Inc. | Cat#CT04 |
| CCG-1423 | Medchem Express | Cat#HY-13991 |
| Cytochalasin B | Cayman Chemical | Cat#11328 |
| KI16425 | Selleck Chem | Cat#S1315 |
| Rho Activator II | Cytoskeleton, Inc. | Cat#CN03 |
| UCM-05194 | Cayman Chemical | Cat#41682 |
| Y27632 | Chemdea | Cat#CD0141 |
| Lysphosphatidic acid | Sigma | Cat#L7260 |
| Cardiolipin | Sigma | Cat#C0563 |
| Lysophosphatidylcholine | Sigma | Cat#L4129 |
| Lysophosphatidylethanolamine | Sigma | Cat#L4754 |
| Phosphatidic Acid | Sigma | Cat#P9511 |
| Phosphatidylcholine | Sigma | Cat#P3556 |
| Phosphatidylethanolamine | Sigma | Cat#P7943 |
| Phosphatidylinositol | Sigma | Cat#P0639 |
| Phosphatidyl-L-serine | Sigma | Cat#P7769 |
| Sphingosine-1-phosphate | Cayman Chemical | Cat#22498 |

Experimental models: Cell lines

| | | |
|---|---|---|
| WTC-mEGFP-TJP1-c120 (mono- | Coriell Institute | ID#AICS-0023 |
| allelic tag) | Biorepository | RRID: CVCL_JM18 |
| Human iPSC 802-3G | ReproCell (via | CAT#RPChiPS8023G1 |
| | Synthego) | |
| SHROOM3-KO isogenic WT iPSC line | Takla et al, 2023¹⁷ | N/A |
| iPSC72.3 | Cellosaurus⁴² | Cat#CCHMCi001-A; |
| | | RRID: CVCL_A1BW |
| Colon-88 patient-derived colon | Dame et al, 2018⁴³ | N/A |
| organoid line | | |
| HT617 patient-derived airway | Gift of Life, 20 | N/A |
| organoid line | yo female | |

Software and algorithms

| | | |
|---|---|---|
| CellPose | Stringer et al, 2021⁴⁴ | www.cellpose.com |
| CellProfiler | Kamentsky et al, | Cellprofiler.org |
| | 2011⁴⁵ | |
| Prism 10 | GraphPad Software | Graphpad.com |
| JMP 17 | JMP | JMP.com |

Having now fully described the invention, it will be understood by those of skill in the art that the same can be performed within a wide and equivalent range of conditions, formulations, and other parameters without affecting the scope of the invention or any embodiment thereof. All patents, patent applications and publications cited herein are fully incorporated by reference herein in their entirety.

### INCORPORATION BY REFERENCE

The entire disclosure of each of the patent documents and scientific articles referred to herein is incorporated by reference for all purposes.

The following references numerically denoted throughout this application are incorporated by reference in their entireties:
1. Co, J. Y., Margalef-Català, M., Li, X., Mah, A. T., Kuo, C. J., Monack, D. M., and Amieva, M. R. (2019). Controlling epithelial polarity: a human enteroid model for host-pathogen interactions. Cell reports 26, 2509-2520. e2504.
2. Salahudeen, A. A., Choi, S. S., Rustagi, A., Zhu, J., van Unen, V., de la O, S. M., Flynn, R. A., Margalef-Català, M., Santos, A. J., and Ju, J. (2020). Progenitor identification and SARS-COV-2 infection in human distal lung organoids. Nature 588, 670-675.
3. Lukovic, D., Artero Castro, A., Kaya, K. D., Munezero, D., Gieser, L., Davó-Martínez, C., Corton, M., Cuenca, N., Swaroop, A., and Ramamurthy, V. (2020). Retinal organoids derived from hiPSCs of an AIPL1-LCA patient maintain cytoarchitecture despite reduced levels of mutant AIPL1. Scientific Reports 10, 5426.
4. Wang, A. Z., Ojakian, G. K., and Nelson, W. J. (1990). Steps in the morphogenesis of a polarized epithelium: II. Disassembly and assembly of plasma membrane domains during reversal of epithelial cell polarity in multicellular epithelial (MDCK) cysts. Journal of cell science 95, 153-165.
5. Akhtar, N., and Streuli, C. H. (2013). An integrin-ILK-microtubule network orients cell polarity and lumen formation in glandular epithelium. Nature cell biology 15, 17-27.
6. Tidball, A. M., Niu, W., Ma, Q., Takla, T. N., Walker, J. C., Margolis, J. L., Mojica-Perez, S. P., Sudyk, R., Deng, L., and Moore, S. J. (2023). Deriving early single-rosette brain organoids from human pluripotent stem cells. Stem Cell Reports.
7. Eiraku, M., Watanabe, K., Matsuo-Takasaki, M., Kawada, M., Yonemura, S., Matsumura, M., Wataya, T., Nishiyama, A., Muguruma, K., and Sasai, Y. (2008). Self-organized formation of polarized cortical tissues from ESCs and its active manipulation by extrinsic signals. Cell stem cell 3, 519-532.
8. Watanabe, M., Buth, J. E., Vishlaghi, N., de la Torre-Ubieta, L., Taxidis, J., Khakh, B. S., Coppola, G., Pearson, C. A., Yamauchi, K., and Gong, D. (2017). Self-organized cerebral organoids with human-specific features predict effective drugs to combat Zika virus infection. Cell reports 21, 517-532.
9. Kadoshima, T., Sakaguchi, H., Nakano, T., Soen, M., Ando, S., Eiraku, M., and Sasai, Y. (2013). Self-organization of axial polarity, inside-out layer pattern, and species-specific progenitor dynamics in human ES cell-derived neocortex. Proceedings of the National Academy of Sciences 110, 20284-20289.
10. Medelnik, J.-P., Roensch, K., Okawa, S., Del Sol, A., Chara, O., Mchedlishvili, L., and Tanaka, E. M. (2018). Signaling-dependent control of apical membrane size and self-renewal in rosette-stage human neuroepithelial stem cells. Stem cell reports 10, 1751-1765.
11. Gotoh, L., Yamada, M., Hattori, K., Sasayama, D., Noda, T., Yoshida, S., Kunugi, H., and Yamada, M. (2019). Levels of lysophosphatidic acid in cerebrospinal fluid and plasma of patients with schizophrenia. Psychiatry Research 273, 331-335.
12. Xu, F., Deng, C., Ren, Z., Sun, L., Meng, Y., Liu, W., Wan, J., and Chen, G. (2021). Lysophosphatidic acid shifts metabolic and transcriptional landscapes to induce a distinct cellular state in human pluripotent stem cells. Cell Reports 37.
13. Lee, J.-H., Shin, H., Shaker, M. R., Kim, H. J., Park, S.-H., Kim, J. H., Lee, N., Kang, M., Cho, S., and Kwak, T. H. (2022). Production of human spinal-cord organoids recapitulating neural-tube morphogenesis. Nature Biomedical Engineering 6, 435-448.
14. Yamashita, A., Kawagishi, N., Miyashita, T., Nagatsuka, T., Sugiura, T., Kume, K., Shimizu, T., and Waku, K. (2001). ATP-independent fatty acyl-coenzyme A synthesis from phospholipid: coenzyme A-dependent transacylation activity toward lysophosphatidic acid catalyzed by acyl-coenzyme A: lysophosphatidic acid acyltransferase. Journal of Biological Chemistry 276, 26745-26752.
15. Yonemura, S., Itoh, M., Nagafuchi, A., and Tsukita, S. (1995). Cell-to-cell adherens junction formation and actin filament organization: similarities and differences between non-polarized fibroblasts and polarized epithelial cells. Journal of cell science 108, 127-142.
16. Contos, J. J., Fukushima, N., Weiner, J. A., Kaushal, D., and Chun, J. (2000). Requirement for the lp A1 lysophosphatidic acid receptor gene in normal suckling behavior. Proceedings of the National Academy of Sciences 97, 13384-13389.
17. Takla, T. N., Luo, J., Sudyk, R., Huang, J., Walker, J. C., Vora, N. L., Sexton, J. Z., Parent, J. M., and Tidball, A. M. (2023). A shared pathogenic mechanism for valproic acid and SHROOM3 knockout in a brain organoid model of neural tube defects. Cells 12, 1697.
18. Kułakowska, A., Byfield, F. J., Żendzian-Piotrowska, M., Zajkowska, J. M., Drozdowski, W., Mroczko, B., Janmey, P. A., and Bucki, R. (2014). Increased levels of sphingosine-1-phosphate in cerebrospinal fluid of patients diagnosed with tick-borne encephalitis. Journal of Neuroinflammation 11, 1-9.
19. Kułakowska, A., Żendzian-Piotrowska, M., Baranowski, M., Konończuk, T., Drozdowski, W., Górski, J., and Bucki, R. (2010). Intrathecal increase of sphingosine 1-phosphate at early stage multiple sclerosis. Neuroscience letters 477, 149-152.
20. Gu, J. L., Müller, S., Mancino, V., Offermanns, S., and Simon, M. I. (2002). Interaction of Gα12 with Gα13 and Gαq signaling pathways. Proceedings of the National Academy of Sciences 99, 9352-9357.
21. McDonald, W. S., Miyamoto, K., Rivera, R., Kennedy, G., Almeida, B. S., Kingsbury, M. A., and Chun, J. (2020). Altered cleavage plane orientation with increased genomic aneuploidy produced by receptor-mediated lysophosphatidic acid (LPA) signaling in mouse cerebral cortical neural progenitor cells. Molecular Brain 13, 1-14.
22. Childs, C. J., Poling, H. M., Chen, K., Tsai, Y.-H., Wu, A., Sweet, C. W., Vallie, A., Eiken, M. K., Huang, S., and Schreiner, R. (2023). Coordinated differentiation of human intestinal organoids with functional enteric neurons and vasculature. bioRxiv, 2023.2011. 2006.565830.
23. Pitstick, A. L., Poling, H. M., Sundaram, N., Lewis, P. L., Kechele, D. O., Sanchez, J. G., Scott, M. A., Broda, T. R., Helmrath, M. A., and Wells, J. M. (2022). Aggregation of cryopreserved mid-hindgut endoderm for more reliable and reproducible hPSC-derived small intestinal organoid generation. Stem Cell Reports 17, 1889-1902.
24. Yu, W., Shewan, A. M., Brakeman, P., Eastburn, D. J., Datta, A., Bryant, D. M., Fan, Q. W., Weiss, W. A., Zegers, M. M., and Mostov, K. E. (2008). Involvement of RhoA, ROCK I and myosin II in inverted orientation of epithelial polarity. EMBO reports 9, 923-929.
25. Yonemura, S. (2014). Differential sensitivity of epithelial cells to extracellular matrix in polarity establishment. PloS one 9, e112922.
26. Onuma, K., Sato, Y., Okuyama, H., Uematsu, H., Homma, K., Ohue, M., Kondo, J., and Inoue, M. (2021). Aberrant activation of Rho/ROCK signaling in impaired polarity switching of colorectal micropapillary carcinoma. The Journal of Pathology 255, 84-94.
27. Offermanns, S., Mancino, V., Revel, J.-P., and Simon, M. I. (1997). Vascular system defects and impaired cell chemokinesis as a result of Gα13 deficiency. Science 275, 533-536.
28. Fotopoulou, S., Oikonomou, N., Grigorieva, E., Nikitopoulou, I., Paparountas, T., Thanassopoulou, A., Zhao, Z., Xu, Y., Kontoyiannis, D. L., and Remboutsika, E. (2010). ATX expression and LPA signalling are vital for the development of the nervous system. Developmental biology 339, 451-464.
29. Katayama, K.-i., Melendez, J., Baumann, J. M., Leslie, J. R., Chauhan, B. K., Nemkul, N., Lang, R. A., Kuan, C.-Y., Zheng, Y., and Yoshida, Y. (2011). Loss of RhoA in neural progenitor cells causes the disruption of adherens junctions and hyperproliferation. Proceedings of the National Academy of Sciences 108, 7607-7612.
30. Escuin, S., Vernay, B., Savery, D., Gurniak, C.B., Witke, W., Greene, N. D., and Copp, A. J. (2015). Rho-kinase-dependent actin turnover and actomyosin disassembly are necessary for mouse spinal neural tube closure. J Cell Sci 128, 2468-2481.
31. Liu, J., Sugimoto, K., Cao, Y., Mori, M., Guo, L., and Tan, G. (2020). Serum sphingosine 1-phosphate (S1P): a novel diagnostic biomarker in early acute ischemic stroke. Frontiers in Neurology 11, 985.
32. Mizugishi, K., Yamashita, T., Olivera, A., Miller, G. F., Spiegel, S., and Proia, R. L. (2005). Essential role for sphingosine kinases in neural and vascular development. Molecular and cellular biology 25, 11113-11121.
33. Fukui, M., Tsutsumi, T., Yamamoto-Mikami, A., Morito, K., Tanaka, T., Iwasa, T., Kuwahara, A., Irahara, M., and Tokumura, A. (2020). Distinct contributions of two choline-producing enzymatic activities to lysophosphatidic acid production in human amniotic fluid from pregnant women in the second trimester and after parturition. Prostaglandins & Other Lipid Mediators 150, 106471.
34. Im Kim, J., Jo, E. J., Lee, H.-Y., Cha, M. S., Min, J. K., Choi, C. H., Lee, Y. M., Choi, Y.-A., Baek, S.-H., and Ryu, S. H. (2003). Sphingosine 1-phosphate in amniotic fluid modulates cyclooxygenase-2 expression in human amnion-derived WISH cells. Journal of Biological Chemistry 278, 31731-31736.
35. Zhang, H., Abraham, N., Khan, L. A., Hall, D. H., Fleming, J. T., and Göbel, V. (2011). Apicobasal domain identities of expanding tubular membranes depend on glycosphingolipid biosynthesis. Nature cell biology 13, 1189-1201.
36. Hu, H.-B., Song, Z.-Q., Song, G.-P., Li, S., Tu, H.-Q., Wu, M., Zhang, Y.-C., Yuan, J.-F., Li, T.-T., and Li, P.-Y. (2021). LPA signaling acts as a cell-extrinsic mechanism to initiate cilia disassembly and promote neurogenesis. Nature Communications 12, 662.
37. Geraldo, L. H. M., Spohr, T. C. L. d. S., Amaral, R. F. d., Fonseca, A. C. C. d., Garcia, C., Mendes, F. d. A., Freitas, C., dosSantos, M. F., and Lima, F. R. S. (2021). Role of lysophosphatidic acid and its receptors in health and disease: novel therapeutic strategies. Signal transduction and targeted therapy 6, 45.
38. Yukiura, H., Kano, K., Kise, R., Inoue, A., and Aoki, J. (2015). Autotaxin overexpression causes embryonic lethality and vascular defects. PLOS One 10, e0126734.
39. Yung, Y. C., Mutoh, T., Lin, M.-E., Noguchi, K., Rivera, R. R., Choi, J. W., Kingsbury, M. A., and Chun, J. (2011). Lysophosphatidic acid signaling may initiate fetal hydrocephalus. Science translational medicine 3, 99ra87-99ra87.
40. Co, J. Y., Margalef-Català, M., Monack, D. M., and Amieva, M. R. (2021). Controlling the polarity of human gastrointestinal organoids to investigate epithelial biology and infectious diseases. Nature protocols 16, 5171-5192.
41. Boecking, C. A., Walentek, P., Zlock, L. T., Sun, D. I., Wolters, P. J., Ishikawa, H., Jin, B.-J., Haggie, P. M., Marshall, W. F., and Verkman, A. S. (2022). A simple method to generate human airway epithelial organoids with externally orientated apical membranes. American Journal of Physiology-Lung Cellular and Molecular Physiology 322, L420-L437.
42. McCracken, K. W., Catá, E. M., Crawford, C. M., Sinagoga, K. L., Schumacher, M., Rockich, B. E., Tsai, Y.-H., Mayhew, C. N., Spence, J. R., and Zavros, Y. (2014). Modelling human development and disease in pluripotent stem-cell-derived gastric organoids. Nature 516, 400-404.
43. Dame, M. K., Attili, D., McClintock, S. D., Dedhia, P. H., Ouillette, P., Hardt, O., Chin, A. M., Xue, X., Laliberte, J., and Katz, E. L. (2018). Identification, isolation and characterization of human LGR5-positive colon adenoma cells. Development 145, dev153049.
44. Stringer, C., Wang, T., Michaelos, M., and Pachitariu, M. (2021). Cellpose: a generalist algorithm for cellular segmentation. Nature methods 18, 100-106.
45. Kamentsky, L., Jones, T. R., Fraser, A., Bray, M.-A., Logan, D. J., Madden, K. L., Ljosa, V., Rueden, C., Eliceiri, K. W., and Carpenter, A. E. (2011). Improved structure, function and compatibility for CellProfiler: modular high-throughput image analysis software. Bioinformatics 27, 1179-1180.

### EQUIVALENTS

The invention may be embodied in other specific forms without departing from the spirit or essential characteristics thereof. The foregoing embodiments are therefore to be considered in all respects illustrative rather than limiting the invention described herein. Scope of the invention is thus indicated by the appended claims rather than by the foregoing description, and all changes that come within the meaning and range of equivalency of the claims are intended to be embraced therein.

1. A method of forming apical-out organoids in a culture, comprising:
contacting a population of cells with an organoid medium, wherein the organoid medium comprises one or both of lysophosphatidic acid (LPA) and sphingosine-1-phosphate (S1P); differentiating the population of cells into organoids; and culturing the organoids in the organoid medium to obtain apical-out organoids, wherein an apical surface of at least a portion of the apical-out organoids faces away from a core thereof.

2. (canceled)

3. (canceled)

4. The method of claim 1, wherein the population of cells initially contacted with the organoid medium lacks apical-out polarity.

5. The method of claim 1, wherein the population of cells comprise epithelial cells and/or mesenchymal cells.

6. (canceled)

7. (canceled)

8. (canceled)

9. The method of claim 1, wherein the population of cells comprises undifferentiated induced pluripotent stem cells (iPSCs) or human iPSCs.

10. (canceled)

11. The method of claim 1, wherein the organoid medium lacks extracellular matrix (ECM) or ECM protein and/or is serum free.

12. (canceled)

13. The method of claim 1, wherein the population of cells were not cultured in extracellular matrix (ECM).

14. The method of claim 1, wherein culturing the population of cells is under non-adherent conditions.

15. (canceled)

16. (canceled)

17. The method of claim 1, wherein the organoid medium comprises an LPA and/or S1P concentration of between 30 and 300 nM.

18. (canceled)

19. The method of claim 1, wherein the obtained apical-out organoids comprise expression of one or more of: ZO1, F-actin, vimentin, TPX2, PAR3, PALS1, cingulin, myosin-IIb, acetylated tubulin, N-cadherin, e-cadherin, and Arl13b.

20. The method of claim 1, wherein the obtained apical-out organoids are apical-out brain organoids comprising expression of one or more of: nestin, PAX6, and N-cadherin.

21. (canceled)

22. The method of claim 1, further comprising culturing the obtained apical-out organoids in a second organoid medium lacking LPA and S1P, wherein the culturing in the absence of LPA and S1P forms apical-in organoids.

23-25. (canceled)

26. A composition comprising apical-out organoids generated through the method of claim 1.

27-29. (canceled)

30. A method of forming an apical-out organoids in a culture, comprising:
contacting differentiated organoids lacking apical-out polarity with an organoid medium, wherein the organoid medium comprises one or both of LPA and S1P; and culturing the differentiated organoids in the organoid medium to obtain apical-out organoids, wherein an apical surface of at least a portion of the apical-out organoids faces away from a core thereof.

31. The method of claim 30, wherein the differentiated organoids are selected from differentiated brain organoids, differentiated lung organoids, differentiated intestinal organoids, and differentiated colon epithelial organoids.

32. The method of claim 30, wherein the organoid medium:
(i) comprises an LPA and/or S1P concentration of between 30 and 300 nM;
(ii) lacks an ECM or ECM protein;
(iii) is serum free.

33. The method of claim 30, wherein the apical surface of at least 60% of the apical-out organoids faces away from the core thereof.
